(19) United States
(12) Patent Application Publication
(10) Pub. No.: US 2002/0155113 A1
(43) Pub. Date: Oct. 24, 2002

(54) Methods for treating or preventing cardiovascular disorders by modulating metalloprotease function

(76) Inventors: Miyoung Chun, Belmont, MA (US); Uwe Schonbeck, Randolph, MA (US); Peter Libby, Boston, MA (US)

Correspondence Address: LOUIS MYERS, Fish & Richardson P.C., 225 Franklin Street, Boston, MA, 02110-2804, US

(21) Appl. No.: 10/097,683

(22) Filed: Mar. 13, 2002

Related U.S. Application Data
(60) Provisional application No. 60/275,881, filed on Mar. 13, 2001.

(51) Int. Cl. A61K 39/395; A61K 38/39

(52) U.S. Cl. 424/146.1; 514/12

(57) ABSTRACT

The present invention is based on the finding that human atheroma-associated endothelial cells (EC), smooth muscle cells (SMC) and macrophages express insterstitial collagenase MMP-8 in vitro, as well as in atherosclerotic lesions in situ. Thus, the invention features methods of modulating the activity or expression of MMP-8 and methods of inhibiting collagen degradation, particularly type I collagen degradation. The invention also features methods of treating or preventing non-neutrophil-mediated inflammatory conditions, in particular cardiovascular disorders such as atherosclerosis; methods of diagnosing and staging such conditions; and methods of evaluating the efficacy of a treatment for such conditions. Finally, the invention features methods of identifying agents that inhibit MMP-8 expression or activity, which can be used for the treatment of non-neutrophil-mediated inflammatory disorders.

### RELATED APPLICATIONS

This application claims priority to U.S. provision application No. 60/275,881, filed on Mar. 13, 2001, the content of which are incorporated herein by reference.

### FUNDING

Work described herein was supported by grants from the National Heart, Lung and Blood Institute (HL-56985). The U.S. government has certain rights in the invention.

### BACKGROUND OF THE INVENTION

Matrix metalloproteases (“MMPs”) are a family of proteases (enzymes) involved in the degradation and remodeling of connective tissues. Members of this family of endopeptidase enzymes are secreted as proenzymes from various cell types that reside in or are associated with connective tissue, such as fibroblasts, monocytes, macrophages, endothelial cells, and invasive or metastatic tumor cells. MMP expression is stimulated by growth factors and cytokines in the local tissue environment, where these enzymes act to specifically degrade protein components of the extracellular matrix, such as collagen, proteoglycans (protein core), fibronectin and laminin. These ubiquitous extracellular matrix components are present in the linings of joints, interstitial connective tissues, basement membranes and cartilage. The MMP family members share a number of properties, including zinc and calcium dependence, secretion as zymogens, and 40-50% amino acid sequence homology. Eleven metalloenzymes have been well-characterized as MMP's in humans, including three collagenases (interstitial collagenases), three stromelysins, two gelatinases, matrilysin, metalloelastase, and membrane-type MMP.

Interstitial collagenases catalyze the initial and rate-limiting cleavage of native collagen types I, II and III. Collagen is an essential component of the matrix of many tissues, for example, cartilage, bone, tendon and skin, as well as atherosclerotic lesions. Interstitial collagen fibrils resist degradation by most proteinases. The interstitial collagenases I (MMP-1), II (MMP-8), and III (MMP-13) are very specific matrix metalloproteases which can initiate the breakdown of intact, triple-helical collagen. The members of this MMP subfamily can cleave all three α-chains of type I, II, and III collagen at Gly⁷⁷⁵-Leu/Ile⁷⁷⁶, degrading the molecule into one-quarter and three-quarter fragments (Mitchell PG et al. J Clin Invest. 1996; 97: 76 1-8; Krane S M et al. J Biol Chem. 1996; 27 1: 28509-15). MMP-8 preferentially degrades type I collagen, while MMP-1 and MMP-13 preferentially cleave type III and II collagen, respectively (Mitchell P G et al. J Clin Invest. 1996; 97: 76 1-8; Horwitz A L et al. Proc Natl Acad Sci U S A. 1977; 74: 897-901; Hasty K A et al. J Biol Chem. 1987; 262: 10048-52; Welgus H G, et al. J Biol Chem. 1981; 256: 951 1-5). Following this initial limited cleavage, the collagen fragments can unwind, loosing their helical structure, and become susceptible to further degradation by interstitial collagenases as well as other MMPs, including those overexpressed in atheroma, e.g., MMP-2, MMP-3, MMP-9 (Henney A M et al. Proc Natl Acad Sci USA. 1991; 88: 8 154-8; Galis Z, et al. J. Clin. Invest. 1994; 94: 2493-2503; Welgus H G et al. J Biol Chem. 1982; 257: 11534-9; Li Z et al. American Journal of Pathology 1996; 148: 12 1-8).

Considerable evidence supports differential expression of these three interstitial collagenases in physiological, as well as pathological situations. Following MMP-1's description as the protease mediating resolution of the tadpole's tail in 1965, early studies focused on this enzyme's (and subsequently MMP-13's) physiological role in embryonic development, organ morphogenesis, endometrial cycling, bone resorption and growth, and wound healing (Matrisian L M. BioEssays 1992; 14: 455-463; Woessner J J. FASEB J. 1991; 5: 2145-2154). A broad spectrum of cell types, including endothelial cells (EC), smooth muscle cells (SMC), and macrophages, can express both MMP-1 (also referred to as human fibroblast-type collagenase (HFC) or collagenase-1) and MMP-13 (Galis Z S et al. Circ. Res. 1994; 75: 181-189).

Originally cloned from mRNA extracted from peripheral blood leukocytes of a patient with chronic granulocytic leukemia, MMP-8 was dubbed ‘neutrophil collagenase’ (also referred to as human neutrophil-type collagenase (HNC) or collagenase-2) (Hasty K A et al. J Biol Chem. 1990; 265: 1142 1-4). Contrary to most MMP family members, neutrophils synthesize MMP-8 early during granulocyte differentiation and store the latent precursor within special granules, available for release upon chemotactic stimulation (Weiss S J, et al. Science. 1985; 227: 747-9; Mookhtiar K A et al. Biochemistry 1990; 29: 10620-7). Numerous studies have reported a role for MMP-8 in connective tissue turnover in acute inflammatory reactions involving neutrophils. The most recently discovered member of this group of MMPs is human collagenase-3 (MMP-13), which was originally found in breast carcinomas (J. Biol. Chem., 269, 16766-16773) (1994)), but has since shown to be produced by chondrocytes (J. Clin. Invest. 97: 761-768, 1996).

### SUMMARY OF THE INVENTION

The present invention is based, at least in part, on the finding that human atheroma-associated endothelial cells (EC), smooth muscle cells (SMC) and macrophages express insterstitial collagenase MMP-8 in vitro, as well as in atherosclerotic lesions (e.g., vulnerable plaques) in situ. This finding provides new modalities in the treatment and diagnosis of non-neutrophil-mediated inflammatory conditions, and in particular cardiovascular disorders, such as atherosclerosis.

Accordingly, in one aspect, the invention features a method of modulating (e.g., inhibiting) the activity, expression, translation, or processing (e.g., release) of matrix metalloprotease-8 (“MMP-8”). The method includes, contacting one or more of: MMP-8, an MMP-8-expressing cell or tissue, or an activator of MMP-8, with an agent, e.g., an MMP-8 inhibitor, in an amount sufficient to modulate (e.g., inhibit) the activity, expression, translation, or processing of MMP-8. The subject method can be used on cells in culture, e.g. in vitro or ex vivo, or in vivo in a subject, e.g., as part of an in vivo therapeutic or prophylactic protocol.

For in vitro embodiments, MMP-8 can be contacted with the agent by, e.g., forming a mixture, e.g., a reconstituted system, which includes MMP-8 and the agent. In other embodiments, an MMP-8-expressing cell (e.g., a macrophage, an endothelial cell, or a smooth muscle cell), or an MMP-8-expressing tissue (e.g., a cardiovascular tissue or an atheroma-associated tissue) is contacted with the agent, e.g., by adding the agent to the culture medium.

The method can also be performed in vivo in a subject. Preferably, the agent, or a pharmaceutically acceptable composition thereof, is administered to the subject in an amount effective to inhibit the activity, expression, translation, or processing of MMP-8. The method can be used for the treatment of, or prophylactic prevention of, a non-neutrophil-mediated disorder, e.g., a disorder involving aberrant activity of macrophage, endothelial and/or smooth muscle cells (e.g., a cardiovascular disorder, such as atherosclerosis, an endothelial cell disorder, or an inflammatory disorder).

For ex vivo embodiments, the method further includes removing MMP-8 or MMP-8-expressing cells from the subject. For example, blood containing MMP-8 or MMP-8-expressing cells, e.g., MMP-8-expressing macrophages, can be obtained from the subject. MMP-8 or MMP-8-expressing cells can be treated with the agent in an amount effective to inhibit the activity, expression, translation, or processing of MMP-8. Treated MMP-8-expressing cells can then be introduced into the subject.

In a preferred embodiment, the method further includes evaluating MMP-8 nucleic acid or protein expression level or activity in the cell or subject before or after the administration or contacting step. For example, a subject, e.g., a patient having, or at risk of a non-neutrophil-mediated disorder, e.g., a cardiovascular disorder, can be evaluated before or after the agent is administered. If the subject has a level of MMP-8 above a predetermined level, therapy can be begun or continued.

In a preferred embodiment, the MMP-8 is human MMP-8. All forms of MMP-8 (i.e., active and latent forms) can be inhibited. Preferably the agent inhibits the active form of MMP-8.

In a preferred embodiment, the agent decreases the expression, translation, activity or processing (e.g., secretion) of MMP-8, e.g., human MMP-8. In one embodiment, the agent can directly inhibit the activity, expression or processing of MMP-8. For example, the agent can interact with, e.g., bind to, an MMP-8 protein and block or reduce the MMP-8 protease activity, e.g., collagenase activity (e.g., the proteolysis of collagen 1). In other embodiments, the agent can block or reduce expression of MMP-8, e.g., by reducing transcription or translation of MMP-8 mRNA, or reducing the stability of MMP-8 mRNA or protein). In still other embodiments, the agent can block the processing of MMP-8, e.g., the agent can inhibit one or more of: the conversion of MMP-8 from a precursor to active form, or the release or secretion of active or latent forms of MMP-8. Alternatively, the agent can indirectly inhibit MMP-8 by inhibiting the activity or expression of: an upstream MMP-8 activator (e.g., a proinflammatory cytokine, e.g., interleukin-1β (IL-1β) or tumor necrosis factor α (TNFα); a lipopolysaccharide (LPS); a costimulatory signal, e.g., CD40 ligand (CD40L); a myeloperoxidase, which in turn reduces the levels of hypochlorous acid; hypochlorous acid; an enzyme involved in the conversion of MMP-8 from latent to active form, or a downstream MMP activator target; or can increase the activity or expression of an MMP-8 inhibitor, or a downstream MMP-8 inhibitor target.

In a preferred embodiment, the agent is a small molecule (e.g., a chemical agent having a molecular weight of less than 2500 Da, preferably, less than 1500 Da), a chemical, e.g., a small organic molecule, e.g., a product of a combinatorial or natural product library; a polypeptide (e.g., an antibody, such as an MMP-8 specific antibody); a peptide, a peptide fragment (e.g., a substrate fragment such as a collagen I fragment), or a peptidomimetic; a modulator (e.g., an inhibitor) of the expression or translation of an MMP-8 nucleic acid, such as a double-stranded RNA (dsRNA) molecule, an antisense molecule, a ribozyme, a triple helix molecule, or any combination thereof.

Preferably, the agent is an MMP-8 specific inhibitor. Examples of MMP-8 specific inhibitors include, but are not limited to, a small molecule MMP-8-specific inhibitor, e.g., a malonic acid-based inhibitor of MMP-8 (e.g., a bis-substituted malonic acid hydroxamate derivative); and an anti-MMP-8 antibody (e.g., a humanized, chimeric, human, or other recombinant (e.g., phage display) anti-MMP-8 antibody).

In other embodiments, the agent is a non-specific MMP inhibitor (i.e., it inhibits two or more MMP's). Examples of non-specific MMP inhibitors include, but are not limited to, a hydroxamic acid, a hydroxamate inhibitor, a carboxylic acid inhibitor, and monoamine derivatives of substituted succinic acids.

In a preferred embodiment, the MMP-8-expressing cell or tissue is an atheroma-associated cell or tissue, e.g., a human atheroma-associated cell or tissue. Preferably, the atheroma-associated cell or tissue is an endothelial cell or tissue, a smooth muscle cell or tissue, or a monocyte or macrophage. In in vivo embodiments, the cell or tissue is associated with (e.g., located in or nearby) an atherosclerotic lesion or plaque, e.g., an early, intermediate or advanced atherosclerotic lesion or plaque. In a particularly preferred embodiment, the cell or tissue is associated with (e.g., located in or nearby) an advanced or rupture-prone atherosclerotic lesion.

Examples of cardiovascular disorders (e.g., inflammatory disorders) that can be treated or prevented with the methods of the invention include, but are not limited to, atherosclerosis, myocardial infarction, stroke, thrombosis, aneurism, heart failure, ischemic heart disease, angina pectoris, sudden cardiac death, hypertensive heart disease; non-coronary vessel disease, such as arteriolosclerosis, small vessel disease, nephropathy, hypertriglyceridemia, hypercholesterolemia, hyperlipidemia, xanthomatosis, asthma, hypertension, emphysema and chronic pulmonary disease; or a cardiovascular condition associated with interventional procedures (“procedural vascular trauma”), such as restenosis following angioplasty, placement of a shunt, stent, synthetic or natural excision grafts, indwelling catheter, valve or other implantable devices. Preferred cardiovascular disorders include atherosclerosis, myocardial infarction, aneurism, and stroke.

In a preferred embodiment, the cardiovascular disorder is caused by aberrant lipid (e.g., fatty acid) metabolism. Examples of disorders involving aberrant lipid metabolism include, but are not limited to, atherosclerosis, arteriolosclerosis, hypertriglyceridemia, obesity, diabetes, hypercholesterolemia, xanthomatosis, and hyperlipidemia. Most preferable, the disorder is atherosclerosis.

In other preferred embodiments, the MMP-8-expressing cell is a macrophage, e.g., a monocyte-derived macrophage. Since macrophages are involved in non-neutrophil mediated inflammatory conditions (e.g., chronic inflammatory conditions), the methods of the invention also encompass non-neutrophil mediated-inflammatory disorders, including but not limited to, an autoimmune disease (e.g., rheumatoid arthritis, allergy, multiple sclerosis, autoimmune diabetes, autoimmune uveitis and nephrotic syndrome), an infectious disease, a malignancy, transplant rejection or graft-versus-host disease, a pulmonary disorder (e.g., chronic obstructive pulmonary disease (COPD)), inflammatory bowel disease (IBD), a bone disorder, an intestinal disorder, or a cardiovascular or an endothelial cell disorder, as described herein.

In other embodiments, the MMP-8 expressing cell is an endothelial cell. Therefore, the methods of the invention can be used to treat, prevent and/or diagnose an endothelial cell mediated disorder, e.g., a disorder involving aberrant proliferation, migration, angiogenesis, or vascularization; or aberrant expression of cell surface adhesion molecules or genes associated with angiogenesis, e.g., TIE-2, FLT and FLK. Endothelial cell disorders include tumorigenesis, tumor metastasis, psoriasis, diabetic retinopathy, endometriosis, Grave's disease, ischemic disease (e.g., atherosclerosis), and chronic inflammatory diseases (e.g., rheumatoid arthritis).

In a preferred embodiment, the subject is a human suffering from, or at risk of, an MMP-8-mediated disorder or disease, e.g., a cardiovascular disorder, a non-neutrophil-mediated disorder, or an endothelial cell disorder, as described herein. For example, the subject is a patient undergoing a therapeutic or prophylactic protocol.

In a preferred embodiment, the subject is a human suffering from, or at risk of, atherosclerosis. For example, a human with early, intermediate or advanced atherosclerosis. Preferably, the subject is a human suffering from, or at risk of, rupture of an atherosclerostic plaque.

In other embodiments, the subject is a non-human animal, e.g., an experimental animal.

The agent(s) described herein can be administered by themselves, or in combination with at least one more agent (referred to herein as a “second agent(s)”), or procedures. In one embodiment, an MMP-8 specific agent is administered in combination with a non-specific matrix metalloprotease inhibitor, e.g., a hydroxamic acid, a hydroxamate inhibitor, a carboxylic acid inhibitor, or a monoamine derivative of substituted succinic acid.

In other embodiments, the agents of the invention can be administered alone or in combination with a cholesterol-lowering agent. Examples of cholesterol lowering agents include bile acid sequestering resins (e.g. colestipol hydrochloride or cholestyramine), fibric acid derivatives (e.g. clofibrate, fenofibrate, or gemfibrozil), thiazolidenediones (e.g., troglitazone, pioglitazone, ciglitazone, englitazone, rosiglitazone), or hydroxymethylglutaryl coenzyme A reductase (HMG-CoA reductase) inhibitors (e.g. statins, such as fluvastatin sodium, lovastatin, pravastatin sodium, simvastatin, atorvastatin calcium, cerivastatin), an ApoAII-lowering agent, a VLDL lowering agent, an ApoAI-stimulating agent, as well as inhibitors of, nicotinic acid, niacin, or probucol. Preferred cholesterol lowering agents include inhibitors of HMG-CoA reductase (e.g., statins), nicotinic acid, and niacin. Preferably, the cholesterol lowering agent results in a favorable plasma lipid profile (e.g., increased HDL and/or reduced LDL).

In other embodiments, the agents of the invention can be administered to a subject in combination with an inflammatory agent that is being used to treat an unrelated disorder, e.g., a viral infection or a cellular proliferation or differentiation disorder such as cancer, wherein treatment of the disorder could increase the risk that the subject will develop a cardiovascular disorder, an endothelial cell disorder, or a non-neutrophil mediated inflammatory disorder. Examples of such inflammatory agents include, but are not limited to, interleukins, e.g., IL-1, IL-2, IL-4, IL-6, or IL-12, interferons alpha or gamma, or immune cell growth factors, e.g., GM-CSF.

In other embodiments, the agent(s) of the invention is administered in combination with an interventional procedure (“procedural vascular trauma”). Examples of interventional procedures, include but are not limited to, angioplasty, placement of a shunt, stent, synthetic or natural excision grafts, indwelling catheter, valve and other implantable devices.

The second agent or procedure can be administered or effected prior to, at the same time, or after administration of the agent(s) of the invention, in single or multiple administration schedules. For example, the second agent and the agents of the invention can be administered continually over a preselected period of time, or administered in a series of spaced doses, i.e., intermittently, for a period of time.

In a preferred embodiment, the agent of the invention, alone or in combination with the second agent or procedure, inhibit (block, reduce or prevent) one or more of: atherosclerotic lesion formation, development or rupture; lipid accumulation and increased plaque stability; collagenolysis, e.g., degradation of type I, II, or III, preferably type I collagen, or the breakdown of intact, triple helical collagen; or the rupture of atherosclerotic plaques.

In a preferred embodiment, the method further includes removing from the subject MMP-8 or MMP-8-expressing cells (e.g., macrophages, endothelial cells or smooth muscle cells), e.g., by separating the MMP-8 or the MMP-8-expressing cells.

In still another aspect, the invention features a method of inhibiting collagen (e.g., collagen I) degradation, in a subject. The method includes administering to the subject an agent that inhibits the activity, expression, translation, or processing of MMP-8, e.g., an agent as described herein, in an amount effective to reduce or inhibit collagen degradation.

In a preferred embodiment, the method further includes evaluating MMP-8, nucleic acid or protein expression level or activity in the subject before or after the administration step. For example, a subject, e.g., a patient at risk of atherosclerotic plaque rupture, can be evaluated before or after the agent is administered. If the subject has a level of MMP-8 above a predetermined level, therapy can begin or be continued.

In a preferred embodiment, the inhibition of collagen degradation is localized to an atherosclerotic lesion or plaque, e.g., an early, intermediate or advanced atherosclerotic lesion or plaque. In one preferred embodiment, the inhibition of collagen degradation is localized to an advanced or rupture-prone atherosclerotic lesion.

In a preferred embodiment, the MMP-8 is human MMP-8.

In one embodiment, the agent can directly inhibit the activity, expression, translation or processing of MMP-8. For example, the agent can interact with, e.g., bind to, an MMP-8 protein and block or reduce the MMP-8 protease activity, e.g., collagenase activity (e.g., the proteolysis of collagen I). In other embodiments, the agent can block or reduce expression of MMP-8, e.g., by reducing transcription or translation of MMP-8 mRNA, or reducing the stability of MMP-8 mRNA or protein). In still other embodiments, the agent can block the processing of MMP-8, e.g., the agent can inhibit one or more of: the conversion of MMP-8 from a precursor to active form, or the release or secretion of active or latent forms of MMP-8. Alternatively, the agent can indirectly inhibit MMP-8 by inhibiting the activity or expression of: an upstream MMP-8 activator (e.g., a proinflammatory cytokine, e.g., interleukin-1β (IL-1β) or tumor necrosis factor α (TNFα); a lipopolysaccharide (LPS); a costimulatory signal, e.g., CD40 ligand (CD40L); a myeloperoxidase, which in turn reduces the levels of hypochlorous acid; hypochlorous acid; an enzyme involved in the conversion of MMP-8 from latent to active form, or a downstream MMP activator target; or can increase the activity or expression of an MMP-8 inhibitor, or a downstream MMP-8 inhibitor target.

In a preferred embodiment, the agent is a small molecule (e.g., a chemical agent having a molecular weight of less than 2500 Da, preferably, less than 1500 Da), a chemical, e.g., a small organic molecule, e.g., a product of a combinatorial or natural product library; a polypeptide (e.g., an antibody, such as an MMP-8 specific antibody); a peptide, a peptide fragment (e.g., a substrate fragment such as a collagen I fragment), or a peptidomimetic; a modulator (e.g., an inhibitor) of the expression or translation of an MMP-8 nucleic acid, such as a double-stranded RNA (dsRNA) molecule, an antisense molecule, a ribozyme, a triple helix molecule, or any combination thereof.

Preferably, the agent is an MMP-8 specific inhibitor. Examples of MMP-8 specific inhibitors include, but are not limited to, a small molecule MMP-8-specific inhibitor, e.g., a malonic acid-based inhibitor of MMP-8 (e.g., a bis-substituted malonic acid hydroxamate derivative); and an anti-MMP-8 antibody (e.g., a humanized, chimeric, human, or other recombinant (e.g., phage display) anti-MMP-8 antibody).

In other embodiments, the agent is a non-specific MMP inhibitor (i.e., it inhibits two or more MMP's). Examples of non-specific MMP inhibitors include, but are not limited to, a hydroxamic acid, a hydroxamate inhibitor, a carboxylic acid inhibitor, and monoamine derivatives of substituted succinic acids.

In a preferred embodiment, the subject is a human suffering from, or at risk of, an MMP-8-mediated disorder or disease, e.g., a cardiovascular disorder, a non-neutrophil-mediated disorder, or an endothelial cell disorder, as described herein. For example, the subject is a patient undergoing a therapeutic or prophylactic protocol.

In a preferred embodiment, the subject is a human suffering from, or at risk of, atherosclerosis. For example, a human with early, intermediate or advanced atherosclerosis. Preferably, the subject is a human suffering from, or at risk of, the rupture of an atherosclerostic plaque.

In other embodiments, the subject is a non-human animal, e.g., an experimental animal.

The agent(s) described herein can be administered by themselves, or in combination with at least one more agent (referred to herein as a “second agent(s)”), or procedures. In one embodiment, an MMP-8 specific agent is administered in combination with a non-specific matrix metalloprotease inhibitor, e.g., a hydroxamic acid, a hydroxamate inhibitor, a carboxylic acid inhibitor, and a monoamine derivative of substituted succinic acid.

In yet other embodiments, the agents of the invention can be administered alone or in combination with a cholesterol-lowering agent. Examples of cholesterol lowering agents include bile acid sequestering resins (e.g. colestipol hydrochloride or cholestyramine), fibric acid derivatives (e.g. clofibrate, fenofibrate, or gemfibrozil), thiazolidenediones (e.g., troglitazone, pioglitazone, ciglitazone, englitazone, rosiglitazone), or hydroxymethylglutaryl coenzyme A reductase (HMG-CoA reductase) inhibitors (e.g. statins, such as fluvastatin sodium, lovastatin, pravastatin sodium, simvastatin, atorvastatin calcium, cerivastatin), an ApoAII-lowering agent, a VLDL lowering agent, an ApoAI-stimulating agent, as well as inhibitors of, nicotinic acid, niacin, or probucol. Preferred cholesterol lowering agents include inhibitors of HMG-CoA reductase (e.g., statins), nicotinic acid, and niacin. Preferably, the cholesterol lowering agent results in a favorable plasma lipid profile (e.g., increased HDL and/or reduced LDL).

In other embodiments, the agents of the invention can be administered to a subject in combination with an inflammatory agent that is being used to treat an unrelated disorder, e.g., a viral infection or a cellular proliferation or differentiation disorder such as cancer, wherein treatment of the disorder could increase the risk that the subject will develop a cardiovascular disorder, an endothelial cell disorder, or a non-neutrophil mediated inflammatory disorder. Examples of such inflammatory agents include, but are not limited to, interleukins, e.g., IL-1, IL-2, IL-4, IL-6, or IL-12, interferons alpha or gamma, or immune cell growth factors, e.g., GM-CSF.

In other embodiments, the agent(s) of the invention is administered in combination with an interventional procedure (“procedural vascular trauma”). Examples of interventional procedures include but are not limited to, angioplasty, placement of a shunt, stent, synthetic or natural excision grafts, indwelling catheter, valve and other implantable devices.

The second agent or procedure can be administered or effected prior to, at the same time, or after administration of the agent(s) of the invention, in single or multiple administration schedules. For example, the second agent and the agents of the invention can be administered continually over a preselected period of time, or administered in a series of spaced doses, i.e., intermittently, for a period of time.

In a preferred embodiment, the agent of the invention, alone or in combination with the second agent or procedure, inhibit (block, reduce or prevent) one or more of: atherosclerotic lesion formation, development or rupture; lipid accumulation and increased plaque stability; collagenolysis, e.g., degradation of type I, II, or III, preferably type I collagen, or the breakdown of intact, triple helical collagen; or the rupture of atherosclerotic plaques.

In a preferred embodiment, the method further includes removing from the subject MMP-8 or MMP-8-expressing cells (e.g., macrophages, endothelial cells or smooth muscle cells), e.g., by separating the MMP-8 or MMP-8-expressing cells.

In yet another aspect, the invention features a method of treating or preventing a cardiovascular disorder, e.g., a cardiovascular disorder as described herein (e.g., atherosclerosis), in a subject. The method includes administering to the subject an agent that inhibits the activity, processing, translation, or expression of MMP-8, e.g., an agent as described herein, in an amount effective to treat or prevent the cardiovascular disorder.

In a preferred embodiment, the agent inhibits or reduced degradation of a collagen substrate, e.g., collagen I, in an atherosclerotic lesion or plaque. The atherosclerotic lesion or plaque can be an early, intermediate or advanced stage lesion or plaque. Preferably, the atherosclerotic lesion or plaque is an advanced stage, e.g., a rupture-prone lesion. In other embodiments, the agent modulates the activity or expression of an atherosclerotic-associated nucleic acid with a resulting beneficial effect in the subject.

In a preferred embodiment, the method further includes evaluating MMP-8 nucleic acid or protein expression level or activity in the subject before or after the administration step. For example, a subject, e.g., a patient at risk of atherosclerotic plaque rupture, can be evaluated before or after the agent is administered. If the subject has a level of MMP-8 above a predetermined level, therapy can begin or be continued.

In a preferred embodiment, the MMP-8 is human MMP-8.

In one embodiment, the agent can directly inhibit the activity, expression, translation or processing of MMP-8. For example, the agent can interact with, e.g., bind to, an MMP-8 protein and block or reduce the MMP-8 protease activity, e.g., collagenase activity (e.g., the proteolysis of collagen I). In other embodiments, the agent can block or reduce expression of MMP-8, e.g., by reducing transcription or translation of MMP-8 mRNA, or reducing the stability of MMP-8 mRNA or protein). In still other embodiments, the agent can block the processing of MMP-8, e.g., the agent can inhibit one or more of: the conversion of MMP-8 from a precursor to active form, or the release or secretion of active or latent forms of MMP-8. Alternatively, the agent can indirectly inhibit MMP-8 by inhibiting the activity or expression of: an upstream MMP-8 activator (e.g., a proinflammatory cytokine, e.g., interleukin-1β (IL-1β) or tumor necrosis factor α (TNFα); a lipopolysaccharide (LPS); a costimulatory signal, e.g., CD40 ligand (CD40L); a myeloperoxidase, which in turn reduces the levels of hypochlorous acid; hypochlorous acid; an enzyme involved in the conversion of MMP-8 from latent to active form, or a downstream MMP activator target; or can increase the activity or expression of an MMP-8 inhibitor, or a downstream MMP-8 inhibitor target.

In a preferred embodiment, the agent is a small molecule (e.g., a chemical agent having a molecular weight of less than 2500 Da, preferably, less than 1500 Da), a chemical, e.g., a small organic molecule, e.g., a product of a combinatorial or natural product library; a polypeptide (e.g., an antibody, such as an MMP-8 specific antibody); a peptide, a peptide fragment (e.g., a substrate fragment such as a collagen I fragment), or a peptidomimetic; a modulator (e.g., an inhibitor) of the expression or translation of an MMP-8 nucleic acid, such as a double-stranded RNA (dsRNA) molecule, an antisense molecule, a ribozyme, a triple helix molecule, or any combination thereof.

Preferably, the agent is an MMP-8 specific inhibitor. Examples of MMP-8 specific inhibitors include, but are not limited to, a small molecule MMP-8-specific inhibitor, e.g., a malonic acid-based inhibitor of MMP-8 (e.g., a bis-substituted malonic acid hydroxamate derivative); and an anti-MMP-8 antibody (e.g., a humanized, chimeric, human, or other recombinant (e.g., phage display) anti-MMP-8 antibody).

In other embodiments, the agent is a non-specific MMP inhibitor (i.e., it inhibits two or more MMP's). Examples of non-specific MMP inhibitors include, but are not limited to, a hydroxamic acid, a hydroxamate inhibitor, a carboxylic acid inhibitor, and monoamine derivatives of substituted succinic acids.

In a preferred embodiment, the subject is a human suffering from, or at risk of, a cardiovascular disease, e.g., a cardiovascular disease as described herein. In other embodiment, the subject is a human suffering from, or at risk of, a disorder involving aberrant lipid (e.g., fatty acid) metabolism, e.g., a lipid metabolic disorder as described herein. For example, the subject is a patient undergoing a therapeutic or prophylactic protocol.

In a preferred embodiment, the subject is a human suffering from, or at risk of, atherosclerosis. For example, a human with early, intermediate or advanced atherosclerosis. Preferably, the subject is a human suffering from, or at risk of, rupture of an atherosclerostic plaque.

In other embodiments, the subject is a non-human animal, e.g., an experimental animal.

The agent(s) described herein can be administered by themselves, or in combination with at least one more agent (referred to herein as a “second agent(s)”), or procedures. In one embodiment, an MMP-8 specific agent is administered in combination with a non-specific matrix metalloprotease inhibitor, e.g., a hydroxamic acid, a hydroxamate inhibitor, a carboxylic acid inhibitor, and a monoamine derivative of substituted succinic acid.

In yet other embodiments, the agents of the invention can be administered alone or in combination with a cholesterol-lowering agent. Examples of cholesterol lowering agents include bile acid sequestering resins (e.g. colestipol hydrochloride or cholestyramine), fibric acid derivatives (e.g. clofibrate, fenofibrate, or gemfibrozil), thiazolidenediones (e.g., troglitazone, pioglitazone, ciglitazone, englitazone, rosiglitazone), or hydroxymethylglutaryl coenzyme A reductase (HMG-CoA reductase) inhibitors (e.g. statins, such as fluvastatin sodium, lovastatin, pravastatin sodium, simvastatin, atorvastatin calcium, cerivastatin), an ApoAII-lowering agent, a VLDL lowering agent, an ApoAI-stimulating agent, as well as inhibitors of, nicotinic acid, niacin, or probucol. Preferred cholesterol lowering agents include inhibitors of HMG-CoA reductase (e.g., statins), nicotinic acid, and niacin. Preferably, the cholesterol lowering agent results in a favorable plasma lipid profile (e.g., increased HDL and/or reduced LDL).

In other embodiments, the agents of the invention can be administered to a subject in combination with an inflammatory agent that is being used to treat an unrelated disorder, e.g., a viral infection or a cellular proliferation or differentiation disorder such as cancer, wherein treatment of the disorder could increase the risk that the subject will develop a cardiovascular disorder, an endothelial cell disorder, or a non-neutrophil mediated inflammatory disorder. Examples of such inflammatory agents include, but are not limited to, interleukins, e.g., IL-1, IL-2, IL-4, IL-6, or IL-12, interferons alpha or gamma, or immune cell growth factors, e.g., GM-CSF.

In other embodiments, the agent(s) of the invention is administered in combination with an interventional procedure (“procedural vascular trauma”). Examples of interventional procedures, include but are not limited to, angioplasty, placement of a shunt, stent, synthetic or natural excision grafts, indwelling catheter, valve and other implantable devices.

The second agent or procedure can be administered or effected prior to, at the same time, or after administration of the agent(s) of the invention, in single or multiple administration schedules. For example, the second agent and the agents of the invention can be administered continually over a preselected period of time, or administered in a series of spaced doses, i.e., intermittently, for a period of time.

In a preferred embodiment, the agent of the invention, alone or in combination with the second agent or procedure, inhibit (block, reduce or prevent) one or more of: atherosclerotic lesion formation, development or rupture; lipid accumulation and increased plaque stability; collagenolysis, e.g., degradation of type I, II, or III, preferably type I collagen, or the breakdown of intact, triple helical collagen; or the rupture of atherosclerotic plaques.

In a preferred embodiment, the method further includes removing from the subject MMP-8, or MMP-8-expressing cells (e.g., macrophages, endothelial cells or smooth muscle cells), e.g., by separating the MMP-8, or the MMP-8-expressing cells.

In yet another aspect, the invention features a method of treating or preventing a non-neutrophil-mediated disorder, e.g., a non-neutrophil mediated mediated inflammatory disorder as described herein, in a subject. The method includes administering to the subject an agent that inhibits the activity, expression or processing of MMP-8, e.g., an agent as described herein, in an amount effective to treat or prevent the disorder.

In a preferred embodiment, the method further includes evaluating MMP-8 nucleic acid or protein expression level or activity in the subject before or after the administration step. For example, a subject, e.g., a patient at risk of atherosclerotic plaque rupture, can be evaluated before or after the agent is administered. If the subject has a level of MMP-8 above a predetermined level, therapy can begin or be continued.

In a preferred embodiment, the MMP-8 is human MMP-8.

In a preferred embodiment, the subject is a human suffering from, or at risk of developing chronic obstructive pulmonary disease (COPD) or inflammatory bowel disease (IBD).

In a preferred embodiment, the agent decreases the expression, translation, activity or processing (e.g., secretion) of MMP-8, e.g., human MMP-8. In one embodiment, the agent can directly inhibit the activity, expression or processing of MMP-8. For example, the agent can interact with, e.g., bind to, an MMP-8 protein and block or reduce the MMP-8 protease activity, e.g., collagenase activity (e.g., the proteolysis of collagen I). In other embodiments, the agent can block or reduce expression of MMP-8, e.g., by reducing transcription or translation of MMP-8 mRNA, or reducing the stability of MMP-8 mRNA or protein). In still other embodiments, the agent can block the processing of MMP-8, e.g., the agent can inhibit one or more of: the conversion of MMP-8 from a precursor to active form, or the release or secretion of active or latent forms of MMP-8. Alternatively, the agent can indirectly inhibit MMP-8 by inhibiting the activity or expression of: an upstream MMP-8 activator (e.g., a proinflammatory cytokine, e.g., interleukin-1β (IL-1β) or tumor necrosis factor α (TNFα); a lipopolysaccharide (LPS); a costimulatory signal, e.g., CD40 ligand (CD40L); a myeloperoxidase, which in turn reduces the levels of hypochlorous acid; hypochlorous acid; an enzyme involved in the conversion of MMP-8 from latent to active form, or a downstream MMP activator target; or can increase the activity or expression of an MMP-8 inhibitor, or a downstream MMP-8 inhibitor target.

In a preferred embodiment, the agent is a small molecule (e.g., a chemical agent having a molecular weight of less than 2500 Da, preferably, less than 1500 Da), a chemical, e.g., a small organic molecule, e.g., a product of a combinatorial or natural product library; a polypeptide (e.g., an antibody, such as an MMP-8 specific antibody); a peptide, a peptide fragment (e.g., a substrate fragment such as a collagen I fragment), or a peptidomimetic; a modulator (e.g., an inhibitor) of the expression or translation of an MMP-8 nucleic acid, such as a double-stranded RNA (dsRNA) molecule, an antisense molecule, a ribozyme, a triple helix molecule, or any combination thereof.

Preferably, the agent is an MMP-8 specific inhibitor. Examples of MMP-8 specific inhibitors include, but are not limited to, a small molecule MMP-8-specific inhibitor, e.g., a malonic acid-based inhibitor of MMP-8 (e.g., a bis-substituted malonic acid hydroxamate derivative); and an anti-MMP-8 antibody (e.g., a humanized, chimeric, human, or other recombinant (e.g., phage display) anti-MMP-8 antibody).

In other embodiments, the agent is a non-specific MMP inhibitor (i.e., it inhibits two or more MMP's). Examples of non-specific MMP inhibitors include, but are not limited to, a hydroxamic acid, a hydroxamate inhibitor, a carboxylic acid inhibitor, and monoamine derivatives of substituted succinic acids.

In other embodiments, the subject is a non-human animal, e.g., an experimental animal.

The agent(s) described herein can be administered by themselves, or in combination with at least one more agent (referred to herein as a “second agent(s)”), or procedures. In one embodiment, an MMP-8 specific agent is administered in combination with a non-specific matrix metalloprotease inhibitor, e.g., a hydroxamic acid, a hydroxamate inhibitor, a carboxylic acid inhibitor, and a monoamine derivative of substituted succinic acid. In a preferred embodiment, the method further includes removing from the subject MMP-8, or MMP-8-expressing cells (e.g., macrophages, endothelial cells or smooth muscle cells), e.g., by separating the MMP-8 or MMP-8-expressing cells.

In some embodiments, the agents of the invention can be administered to a subject in combination with an inflammatory agent that is being used to treat an unrelated disorder, e.g., a viral infection or a cellular proliferation or differentiation disorder such as cancer, wherein treatment of the disorder could increase the risk that the subject will develop a cardiovascular disorder, an endothelial cell disorder, or a non-neutrophil mediated inflammatory disorder. Examples of such inflammatory agents include, but are not limited to, interleukins, e.g., IL-1, IL-2, IL-4, IL-6, or IL-12, interferons alpha or gamma, or immune cell growth factors, e.g., GM-CSF.

The second agent or procedure can be administered or effected prior to, at the same time, or after administration of the agent(s) of the invention, in single or multiple administration schedules. For example, the second agent and the agents of the invention can be administered continually over a preselected period of time, or administered in a series of spaced doses, i.e., intermittently, for a period of time.

In a preferred embodiment, the method further includes removing from the subject MMP-8, or MMP-8-expressing cells (e.g., macrophages, endothelial cells or smooth muscle cells), e.g., by separating the MMP-8, or the MMP-8-expressing cells.

In yet another aspect, the invention features a method of treating or preventing, in a subject, a disorder characterized by aberrant expression or activity of MMP-8 in a macrophage, an endothelial cell, or a smooth muscle cell. The method includes administering to the subject an agent that inhibits the activity, processing, translation, or expression of MMP-8, e.g., an agent as described herein, in an amount effective to treat or prevent the disorder.

In a preferred embodiment, the method further includes evaluating nucleic acid or protein expression level or activity of MMP-8 in the subject before or after the administration step. If the subject has a level of MMP-8 above a predetermined level, therapy can begin or be continued.

In a preferred embodiment, the MMP-8 is human MMP-8.

In a preferred embodiment, the agent decreases the expression, translation, activity or processing (e.g., secretion) of MMP-8, e.g., human MMP-8. In one embodiment, the agent can directly inhibit the activity, expression or processing of MMP-8. For example, the agent can interact with, e.g., bind to, an MMP-8 protein and block or reduce the MMP-8 protease activity, e.g., collagenase activity (e.g., the proteolysis of collagen I). In other embodiments, the agent can block or reduce expression of MMP-8, e.g., by reducing transcription or translation of MMP-8 mRNA, or reducing the stability of MMP-8 mRNA or protein). In still other embodiments, the agent can block the processing of MMP-8, e.g., the agent can inhibit one or more of: the conversion of MMP-8 from a precursor to active form, or the release or secretion of active or latent forms of MMP-8. Alternatively, the agent can indirectly inhibit MMP-8 by inhibiting the activity or expression of: an upstream MMP-8 activator (e.g., a proinflammatory cytokine, e.g., interleukin-1β (IL-1β) or tumor necrosis factor α (TNFα); a lipopolysaccharide (LPS); a costimulatory signal, e.g., CD40 ligand (CD40L); a myeloperoxidase, which in turn reduces the levels of hypochlorous acid; hypochlorous acid; an enzyme involved in the conversion of MMP-8 from latent to active form, or a downstream MMP activator target; or can increase the activity or expression of an MMP-8 inhibitor, or a downstream MMP-8 inhibitor target.

In a preferred embodiment, the agent is a small molecule (e.g., a chemical agent having a molecular weight of less than 2500 Da, preferably, less than 1500 Da), a chemical, e.g., a small organic molecule, e.g., a product of a combinatorial or natural product library; a polypeptide (e.g., an antibody, such as an MMP-8 specific antibody); a peptide, a peptide fragment (e.g., a substrate fragment such as a collagen I fragment), or a peptidomimetic; a modulator (e.g., an inhibitor) of the expression or translation of an MMP-8 nucleic acid, such as a double-stranded RNA (dsRNA) molecule, an antisense molecule, a ribozyme, a triple helix molecule, or any combination thereof.

Preferably, the agent is an MMP-8 specific inhibitor. Examples of MMP-8 specific inhibitors include, but are not limited to, a small molecule MMP-8-specific inhibitor, e.g., a malonic acid-based inhibitor of MMP-8 (e.g., a bis-substituted malonic acid hydroxamate derivative); and an anti-MMP-8 antibody (e.g., a humanized, chimeric, human, or other recombinant (e.g., phage display) anti-MMP-8 antibody).

In other embodiments, the agent is a non-specific MMP inhibitor (i.e., it inhibits two or more MMP's). Examples of non-specific MMP inhibitors include, but are not limited to, a hydroxamic acid, a hydroxamate inhibitor, a carboxylic acid inhibitor, and monoamine derivatives of substituted succinic acids.

In a preferred embodiment, the subject is a human suffering from, or at risk of, an MMP-8-mediated disorder or disease, e.g., a cardiovascular disorder, a non-neutrophil-mediated disorder (e.g., inflammatory disorder, e.g., COPD or IBD), or an endothelial cell disorder, as described herein. For example, the subject can be a patient undergoing a therapeutic or prophylactic protocol.

In a preferred embodiment, the subject is a human suffering from, or at risk of, atherosclerosis. For example, a human with early, intermediate or advanced atherosclerosis. Preferably, the subject is a human suffering from, or at risk of, rupture of an atherosclerostic plaque.

In other embodiments, the subject is a non-human animal, e.g., an experimental animal.

The agent(s) described herein can be administered by themselves, or in combination with at least one more agent (referred to herein as a “second agent(s)”), or procedures. In one embodiment, an MMP-8 specific agent is administered in combination with a non-specific matrix metalloprotease inhibitor, e.g., a hydroxamic acid, a hydroxamate inhibitor, a carboxylic acid inhibitor, and a monoamine derivative of substituted succinic acid.

In yet other embodiments, the agents of the invention can be administered alone or in combination with a cholesterol lowering agent. Examples of cholesterol lowering agents include bile acid sequestering resins (e.g. colestipol hydrochloride or cholestyramine), fibric acid derivatives (e.g. clofibrate, fenofibrate, or gemfibrozil), thiazolidenediones (e.g., troglitazone, pioglitazone, ciglitazone, englitazone, rosiglitazone), or hydroxymethylglutaryl coenzyme A reductase (HMG-CoA reductase) inhibitors (e.g. statins, such as fluvastatin sodium, lovastatin, pravastatin sodium, simvastatin, atorvastatin calcium, cerivastatin), an ApoAII-lowering agent, a VLDL lowering agent, an ApoAI-stimulating agent, as well as inhibitors of, nicotinic acid, niacin, or probucol. Preferred cholesterol lowering agents include inhibitors of HMG-CoA reductase (e.g., statins), nicotinic acid, and niacin. Preferably, the cholesterol lowering agent results in a favorable plasma lipid profile (e.g., increased HDL and/or reduced LDL).

In other embodiments, the agents of the invention can be administered to a subject in combination with an inflammatory agent that is being used to treat an unrelated disorder, e.g., a viral infection or a cellular proliferation or differentiation disorder such as cancer, wherein treatment of the disorder could increase the risk that the subject will develop a cardiovascular disorder, an endothelial cell disorder, or a non-neutrophil mediated inflammatory disorder. Examples of such inflammatory agents include, but are not limited to, interleukins, e.g., IL-1, IL-2, IL-4, IL-6, or IL-12, interferons alpha or gamma, or immune cell growth factors, e.g., GM-CSF.

In other embodiments, the agent(s) of the invention is administered in combination with an interventional procedure (“procedural vascular trauma”). Examples of interventional procedures, include but are not limited to, angioplasty, placement of a shunt, stent, synthetic or natural excision grafts, indwelling catheter, valve and other implantable devices.

The second agent or procedure can be administered or effected prior to, at the same time, or after administration of the agent(s) of the invention, in single or multiple administration schedules. For example, the second agent and the agents of the invention can be administered continually over a preselected period of time, or administered in a series of spaced doses, i.e., intermittently, for a period of time.

In a preferred embodiment, the agent of the invention, alone or in combination with the second agent or procedure, inhibit (block, reduce or prevent) one or more of: atherosclerotic lesion formation, development or rupture; lipid accumulation and increased plaque stability; collagenolysis, e.g., degradation of type I, II, or III, preferably type I collagen, or the breakdown of intact, triple helical collagen; or the rupture of atherosclerotic plaques.

In a preferred embodiment, the method further includes removing from the subject MMP-8, or MMP-8 expressing cells (e.g., macrophages, endothelial or smooth muscle cells), e.g., by separating the MMP-8, or MMP-8 expressing cells.

The invention also features a method of diagnosing, or staging, an MMP-8-mediated disorder, e.g., a cardiovascular disorder (e.g., atherosclerosis), an endothelial cell disorder, or a non-neutrophil-mediated inflammatory disorder, in a subject. The method includes evaluating the expression, activity or processing, of an MMP-8 nucleic acid or polypeptide, thereby diagnosis or staging the disorder. In a preferred embodiment, the expression or activity is compared with a reference value, wherein a difference, e.g., an increase, in the expression or activity level of the MMP-8 nucleic or polypeptide relative to a normal subject or a cohort of normal subjects is indicative of the disorder, or a stage in the disorder.

In a preferred embodiment, the subject is a human. For example, the subject is a human suffering from, or at risk of, a cardiovascular disorder as described herein. Preferably, subject is a human suffering from, or at risk of, atherosclerosis; a human with early, intermediate or advanced atherosclerosis; or a human suffering from, or at risk of, the rupture of an atherosclerostic plaque. In other embodiments, the subject is a human suffering from, or at risk of, an endothelial cell disorder or a non-neutrophil-mediated inflammatory disorder as described herein.

In a preferred embodiment, the evaluating step occurs in vitro or ex vivo. For example, a sample, e.g., blood, plasma, a tissue sample, or a biopsy, is obtained from the subject. Preferably, the sample contains an MMP-8-expressing cell, e.g., an atheroma-associated cell (e.g., a macrophage, endothelial cell, or smooth muscle cell). In one embodiment, plasma levels of MMP-8 are evaluated by determining, e.g., the level of functional MMP-8 in the plasma. Alternatively, the level of collagen breakdown products present in, e.g., a subject's plasma, can be evaluated.

In a preferred embodiment, the evaluating step occurs in vivo. For example, by administering to the subject a detectably labeled agent that interacts with the MMP-8-associated nucleic acid or polypeptide, such that a signal is generated in an amount proportional to the level of activity or expression of the MMP-8 nucleic acid or polypeptide.

In other preferred embodiments, the method is performed on a sample from a subject, e.g., a human subject, to determine if the individual from which the target nucleic acid or protein is taken should receive a drug or other treatment, to diagnose an individual for a disorder or for predisposition to resistance to treatment, or to stage a disease or disorder. The sample can be from: a subject, e.g., a patient, suffering from, or at risk of, a cardiovascular, endothelial, or non-neutrophil-mediated inflammatory disorder as described herein; a patient suffering from, or at risk of, atherosclerosis (e.g., a human with early, intermediate or advanced atherosclerosis); or a patient suffering from, or at risk of, rupture of an atherosclerostic plaque;

In a preferred embodiment, the level of expression of at least one, two, three or four atherosclerosis-associated nucleic acids or polypeptides is evaluated. Examples of atherosclerosis-associated nucleic acid or polypeptide include, but are not limited to, MMP-1, MMP-8, MMP-13, MMP-14, PAI, PAI-2, and TGF-β. Preferably, the atherosclerosis-associated nucleic acid or polypeptide is MMP-8, most preferably human MMP-8.

In a preferred embodiment, the expression of an atherosclerosis- or MMP-8-associated nucleic acid is evaluated by evaluating the expression of a signal entity, e.g., a green fluorescent protein or leuciferase, which is under the control or an atherosclerosis- or MMP-8-associated gene control element e.g., a promoter, e.g., an MMP-8 promoter.

In some embodiments, the expression of one or more atherosclerosis-associated nucleic acid or polypeptide is evaluated by contacting said sample with, a nucleic acid probe that selectively hybridizes to one or more atherosclerosis-associated nucleic acids or polypeptides. An increase in the level of said one or more atherosclerosis-associated nucleic acids or polypeptides, relative to a control, indicates a disorder, or a stage in the disorder.

In some embodiments, nucleic acid (or protein) from the cell or sample is analyzed on a positional array, e.g., a DNA-chip array. Accordingly, in preferred embodiments the method further includes:

analyzing the sample by providing an array of a plurality of capture probes, wherein each of the capture probes is positionally distinguishable from other capture probes of the plurality on the array, and wherein each positional distinguishable capture probe includes a unique reagent, e.g., an antibody or a nucleic acid probe which can identify an atherosclerosis- or MMP-8-associated nucleic acid or polypeptide; and

hybridizing the sample with the array of capture probes, thereby analyzing the sample sequence.

In a preferred embodiment, the MMP-8-mediated disorder is a cardiovascular disorder, e.g., a cardiovascular disorder as described herein. Preferably, the disorder is atherosclerosis (e.g., early, intermediate or advanced atherosclerosis). Most preferably, the disorder is advanced stage atherosclerosis, e.g., an atherosclerotic stage characterized by rupture-prone atherosclerotic plaques or lesions.

In a preferred embodiment, the MMP-8-mediated disorder is an endothelial disorder, as described herein.

In a preferred embodiment, the MMP-8-mediated disorder is a non-neutrophil-mediated inflammatory disorder, as described herein.

In a further aspect, the invention provides assays for determining the presence or absence of a genetic alteration in an MMP-8 nucleic acid or polypeptide, including for disease diagnosis, a response to cardiovascular therapy.

In a related aspect, the invention provides a method of evaluating a subject, e.g., to identify a predisposition to an MMP-8 mediated disorder (e.g., a cardiovascular, endothelial cell or non-neutrophil mediated inflammatory disorder), diagnose, or treat the subject. The method includes providing a nucleic acid of the subject; and either a) determining the allelic identity of an atherosclerosis (MMP-8)-associated nucleic acid (e.g., MMP-8, preferably, human MMP-8) or b) determining the sequence of at least a nucleotide of the nucleic acid. In a preferred embodiment, the method further includes comparing the allelic identity or sequence to a reference allele or reference sequence of the nucleic acid. The reference allele or reference sequence is associated with an immune disorder or a functional (e.g., normal) immune system. Allelic variants can be detected using, e.g., arrays, mismatch cleavage, electrophoretic assays, HPLC assays, and nucleic acid sequencing. Preferably, the assays detect nucleotide substitutions, and preferably, also insertions, deletions, translocations, and rearrangements of an atherosclerosis (MMP-8)-associated nucleic acid (e.g., MMP-8, preferably, human MMP-8).

In a preferred embodiment, the method further includes diagnosing a subject, and/or choosing a therapeutic modality, e.g., a particular treatment, or a dosage thereof, based on the level of atherosclerosis-associated nucleic acid (e.g., MMP-8) expression or allelic identity.

In another aspect, the invention features, a method for evaluating the efficacy of a treatment of a disorder, e.g., an MMP-8-mediated disorder, e.g., a cardiovascular disorder (e.g., atherosclerosis), an endothelial cell disorder, or a non-neutrophil-mediated inflammatory disorder, in a subject. The method includes evaluating the expression of one or more atherosclerosis-associated nucleic acids or polypeptides, thereby evaluating the efficacy of the treatment. In a preferred embodiment, the expression or activity is compared with a reference value. A change, e.g., decrease, in the level of said one or more atherosclerosis-associated nucleic acids or polypeptides in a sample obtained after treatment, relative to the level of expression before treatment, is indicative of the efficacy of the treatment of said disorder.

In a preferred embodiment, the subject is a human. For example, the subject is a human suffering from, or at risk of, a cardiovascular disorder as described herein. Preferably, subject is a human suffering from, or at risk of, atherosclerosis; a human with early, intermediate or advanced atherosclerosis; or a human suffering from, or at risk of, rupture of an atherosclerostic plaque. In other embodiments, the subject is a human suffering from, or at risk of, a non-neutrophil-mediated inflammatory disorder, or an endothelial disorder, as described herein.

In another preferred embodiment, the subject is an animal, e.g., an experimental animal.

In a preferred embodiment, the evaluating step occurs in vitro or ex vivo. For example, a sample, e.g., blood, plasma, tissue sample, a biopsy, is obtained from the subject. Preferably, the sample contains atheroma-associated cells, e.g., macrophages, endothelial cells, or smooth muscle cells.

For in vitro embodiments, the method includes providing a sample, e.g., a tissue, a bodily fluid (e.g., blood), or a biopsy, from said subject;

evaluating the expression of one or more atherosclerosis-associated nucleic acids or polypeptides, e.g., by contacting the sample with a nucleic acid probe that selectively hybridizes to one or more atherosclerosis-associated nucleic acids, or an antibody that specifically binds to one or more atherosclerosis-associated polypeptides,

wherein a change, e.g., a decrease, in the level of said one or more atherosclerosis-associated nucleic acids or polypeptides in a sample obtained after treatment, relative to the level of expression before treatment, is indicative of the efficacy of the treatment of said disorder.

In preferred embodiments, the method is performed on a sample from a subject, e.g., a human subject. For example, the sample can be obtained from: a patient suffering from, or at risk of, a cardiovascular or non-neutrophil-mediated inflammatory disorder, as described herein; a patient suffering from, or at risk of, atherosclerosis (e.g., a human with early, intermediate or advanced atherosclerosis); or a human suffering from, or at risk of, rupture of an atherosclerostic plaque.

In a preferred embodiment, the atherosclerosis-associated nucleic acid or polypeptide include, but are not limited to, MMP-1, MMP-8, MMP-13, MMP-14, PAI, PAI-2, and TGF-β. Preferably, the atherosclerosis-associated nucleic acid or polypeptide is MMP-8, preferably human MMP-8.

In a preferred embodiment, the sample contains atheroma-associated cells, e.g., macrophages, endothelial cells, or smooth muscle cells.

In a preferred embodiment, the method further includes diagnosis and/or choosing a therapeutic modality, e.g., a particular treatment, or a dosage thereof, based on the level of atherosclerosis-associated nucleic acid expression (e.g., MMP-8 expression).

In a preferred embodiment, the expression of atherosclerosis- or MMP-8-associated nucleic acid is evaluated by evaluating the expression of a signal entity, e.g., a green fluorescent protein or other marker protein, which is under the control or an atherosclerosis- or MMP-8-associated gene control element e.g., a promoter, e.g., an MMP-8 promoter.

In some embodiments, nucleic acid (or protein) from the cell or sample is analyzed on a positional array, e.g., a DNA-chip array. Accordingly, in preferred embodiments the method further includes:

analyzing the sample by providing an array of a plurality of capture probes, wherein each of the capture probes is positionally distinguishable from other capture probes of the plurality on the array, and wherein each positional distinguishable capture probe includes a unique reagent, e.g., an antibody or a nucleic acid probe which can identify an atherosclerosis- or MMP-8-associated nucleic acid or polypeptide;

hybridizing the sample with the array of capture probes, thereby analyzing the sample sequence.

In a preferred embodiment, the evaluating step occurs in vivo. For example, by administering to the subject a detectably labeled agent that interacts with the MMP-8-associated nucleic acid or polypeptide, such that a signal is generated in an amount proportional to the level of activity or expression of the MMP-8 nucleic acid or polypeptide.

In yet another aspect, the invention features a method of selecting a cell (e.g., a macrophage, endothelial cell, or smooth muscle cell) having a selected level of MMP-8 expression or activity, e.g., a cell having a selected level of activated MMP-8.

In a preferred embodiment, the method compares the expression of MMP-8 to a preselected standard, e.g., a control cell. In some embodiments, the expression of MMP-8 is determined directly, e.g., by determining the level of MMP-8 protein or nucleic acid. In other embodiments, the expression of MMP-8 is determined indirectly, e.g., using a GFP reporter construct linked to the MMP-8 promoter.

In a preferred embodiment, the method includes contacting said cell with an agent, e.g., an antibody, that selectively binds to activated forms of MMP-8 relative to latent MMP-8 forms, under conditions that allow binding to occur. In one embodiment, the agent is coupled to, e.g., conjugated with, a moiety that allows separation (e.g., physical separation) of the bound agent-MMP-8 complex. For example, the agent can be an antibody conjugated to a fluorescent or paramagnetic moiety, thereby allowing cells expression MMP-8 to be separated by fluorescence activated cell sorting (FACS) or using magnetic beads, respectively.

In a preferred embodiment, the method includes determining resting from activated cells.

In yet another aspect, the invention features a method of evaluating, or identifying, an agent, e.g., an agent as described herein (e.g., a polypeptide, peptide, a peptide fragment, a peptidomimetic, a small molecule), for the ability to modulate, e.g. inhibit, the activity, processing, translation or expression of an MMP-8 nucleic acid or protein. Such agents are useful for treating or preventing cardiovascular disorders (e.g., atherosclerosis), endothelial cell disorders, or non-neutrophil-mediated inflammatory disorders, as described herein. The method includes:

providing a test agent, an MMP-8 protein or a cell expressing MMP-8 (e.g., an atheroma-associated cell), and an MMP-8 substrate, e.g., collagen (e.g., collagen I);

contacting said test agent, said MMP-8 protein or cell expressing MMP-8, and said MMP-8 substrate, under conditions that allow an interaction between said MMP-8 protein and said MMP-8 substrate to occur; and

determining whether said test agent modulates (e.g., decreases) the interaction between said MMP-8 and said MMP-8 substrate (e.g., reduces cleavage of the MMP-8 substrate),

wherein a change, e.g., a decrease, in the interaction between said MMP-8 protein and said MMP-8 substrate in the presence of the test agent, relative to the interaction in the absence of the test agent, is indicative of modulation, e.g. inhibition, of the activity, processing, translation or expression of an MMP-8 nucleic acid or protein.

In a preferred embodiment, the method further comprises the step of evaluating the test agent in an atheroma-associated cell, e.g., a macrophage, smooth muscle cell or endothelial cell, in vitro, ex vivo, or in vivo (e.g., in a subject, e.g., a patient having atherosclerosis), to thereby determine the effect of the test agent on the expression, translation, processing or activity of the MMP-8.

In a preferred embodiment, the contacting step occurs in vitro or ex vivo. For example, a sample, e.g., a blood sample, is obtained from the subject. Preferably, the sample contains an atheroma-associated cell, e.g., a macrophage, an endothelial cell or a smooth muscle cell.

In a preferred embodiment, the MMP-8 substrate is a fluorogenic substrate, e.g., an FITC-conjugated small peptide. Preferably, the fluorogenic substrate releases fluorescence upon cleavage.

In some embodiments, the MMP-8 substrate may interact with, e.g., bind to, other MMP's, e.g., MMP-2, -9, or -13.

In a preferred embodiment, the contacting step occurs in vivo. For example, by administering to the subject a detectably labeled agent that interacts with the MMP-8 nucleic acid or polypeptide, such that a signal is generated relative to the level of expression, translation, processing or activity of the MMP-8 nucleic acid or polypeptide.

In a preferred embodiment, the test agent is an inhibitor (partial or complete inhibitor) of the MMP-8 polypeptide expression, translation, processing or activity.

In preferred embodiments, the test agent is a peptide, a small molecule, e.g., a member of a combinatorial library (e.g., a peptide or organic combinatorial library, or a natural product library), or an antibody, or any combination thereof.

In additional preferred embodiments, the test agent is a dsRNS molecule (e.g., a 21 base-pair dsRNA molecule), an antisense molecule, a ribozyme, a triple helix molecule, an atherosclerotic-associated nucleic acid, or any combination thereof.

In some embodiments, the test agent may interact with, e.g., bind to, other MMP's, e.g., MMP-2, -9, or -13.

In a preferred embodiment, a plurality of test agents, e.g., library members, is tested. In a preferred embodiment, the plurality of test agents, e.g., library members, includes at least 10, 10², 10³, 10⁴, 10⁵, 10⁶, 10⁷, or 10⁸ compounds. In a preferred embodiment, the plurality of test agents, e.g., library members, share a structural or functional characteristic.

In a preferred embodiment, the test agent is a peptide or a small organic molecule.

In a preferred embodiment, the method is performed in cell-free conditions (e.g., a reconstituted system).

In a preferred embodiment, the method further includes: contacting said agent with a test cell, or a test animal, to evaluate the effect of the test agent on the expression, translation, processing or activity of MMP-8.

In a preferred embodiment, the ability of the agent to modulate the expression, translation, processing or activity of MMP-8 is evaluated in a second system, e.g., a cell-free, cell-based, or an animal system.

In a preferred embodiment, the ability of the agent to modulate the expression, translation, processing or activity of MMP-8 is evaluated in a cell based system, e.g., a two-hybrid assay.

In another aspect, the invention features a method of evaluating, or identifying, an agent, e.g., an agent as described herein (e.g., a polypeptide, peptide, a peptide fragment, a peptidomimetic, a small molecule), for the ability to modulate, e.g. enhance or decrease, the transcription of an atherosclerotic-associated nucleic acid. The method includes:

contacting a cell, e.g., an atheroma-associated cell (e.g., a macrophage or a monocyte, an endothelial cell, or a smooth muscle cell), with a test agent; and

determining whether said test agent modulates, e.g., activates or inhibits, transcription of at least one atherosclerotic-associated nucleic acid,

wherein a change, e.g., an increase or decrease, in the level of expression of said atherosclerotic-associated nucleic acid is indicative of a modulation, e.g., activation or inhibition, of the expression of atherosclerotic-associated nucleic acids.

In a preferred embodiment, the level of expression of at least one, two, three or four atherosclerotic-associated nucleic acid or polypeptide is evaluated. Examples of such nucleic acids or polypeptides include, but are not limited to, MMP-1, MMP-8, MMP-13, MMP-14, PAI, PAI-2, and TGF-β. Preferably, the atherosclerosis-associated nucleic acid or polypeptide is MMP-8, most preferably human MMP-8.

In a preferred embodiment, the level of expression of the at least one atherosclerotic-associated nucleic acid (e.g., a nucleic acid as described herein) is evaluated after stimulation of the cell, e.g., the atheroma-associated cell (e.g., a macrophage or a monocyte), with a proinflammatory agent, e.g., a proinflammatory cytokine (e.g., IL-1β, CD40L, TNFα, or LPS).

In preferred embodiments, the test agent is a peptide, a small molecule, e.g., a member of a combinatorial library (e.g., a peptide or organic combinatorial library, or a natural product library), an antibody, or any combination thereof.

In additional preferred embodiments, the test agent is a dsRNA molecule (e.g., a 21 base-pair dsRNA molecule), an antisense, a ribozyme, a triple helix molecule, an atherosclerotic-associated nucleic acid, or any combination thereof.

In a preferred embodiment, a plurality of test compounds, e.g., library members, is tested. In a preferred embodiment, the plurality of test compounds, e.g., library members, includes at least 10, 10², 10³, 10⁴, 10⁵, 10⁶, 10⁷, or 10⁸ compounds. In a preferred embodiment, the plurality of test compounds, e.g., library members, share a structural or functional characteristic.

In a preferred embodiment, test compound is a peptide or a small organic molecule.

In a preferred embodiment, the method is performed in cell-free conditions (e.g., a reconstituted system).

In a preferred embodiment, the method is performed in a cell, e.g., an atheroma-associated cell (e.g., a macrophage or a monocyte, an endothelial cell or a smooth muscle cell).

In a preferred embodiment, the method further includes: contacting said agent with a test cell, or a test animal, to evaluate the effect of the test agent on the transcription of the atherosclerotic-associated nucleic acid.

In a preferred embodiment, the ability of the agent to modulate transcription of the atherosclerotic-associated nucleic acid is evaluated in a second system, e.g., a cell-free, cell-based, or an animal system.

In a preferred embodiment, the ability of the agent to modulate transcription of the atherosclerotic-associated nucleic acid is evaluated in a cell-based system, using, e.g., a reporter construct, e.g., a construct encoding luciferase or GFP under the control of the promoter of an atherosclerosis-associated nucleic acid, e.g., an MMP-8 promoter.

Also within the scope of the invention are agents identified using the methods described herein.

In another aspect, the invention features a pharmaceutical composition comprising an agent as described herein, and a pharmaceutically acceptable carrier. In one embodiment, the compositions of the invention, e.g., the pharmaceutical compositions, are administered in combination therapy, i.e., combined with other agents, e.g., therapeutic agents, that are useful for treating cardiovascular disorders, such as atherosclerosis. The agent can be in the form of a prodrug, or a pharmaceutically acceptable salt or solvate thereof.

Other features, objects, and advantages of the invention will be apparent from the description and drawings, and from the claims.

### BRIEF DESCRIPTION OF THE DRAWINGS

FIG. 1 is a bar graph depicting expression of MMP-8 mRNA in human monocyte-derived macrophages upon stimulation with atheroma-associated proinflammatory cytokines.

FIG. 2 depicts the colocalization, by double-immunofluorescence staining, of human MMP-8 with human vascular endothelial cells (EC), smooth muscle cells (SMC), and macrophages (MΦ) in atherosclerotic lesions. Analysis of surgical specimens of atheroma from three different donors showed similar results.

FIG. 3 depicts the enhanced expression of human MMP-8 protein in atherosclerotic lesions. Protein extracts (50 μg) obtained from frozen tissue of three donor of non-atherosclerotic carotid arteries (Normal), as well as carotid plaques, dichotomized into lesions characterized by features associated with stable or vulnerable plaques, were analyzed by Western blotting with either anti-MMP-8 antibody alone (left) or MMP-8 antibody preincubated with recombinant MMP-8 (5 mg/ml recMMP-8; right). Positions of molecular weight markers are indicated on left.

FIGS. 4A and B depicts the colocalization of human MMP-8 with cleaved type I collagen in atherosclerotic lesions. In FIG. 4A, collagen was localized to the smooth muscle-enriched region of atherosclerotic lesions (right) using picrosirius red staining (left). In FIG. 4B, immunofluorescence double-labeling was used to colocalized MMP-8 (left panels) with three-quarter-length collagen fragments (top two panels) and to demonstrate an inverse correlation in the distribution of MMP-8 and intact type I collagen within the shoulder region of atherosclerotic plaques (bottom two panels). Analysis of surgical specimens from two different donors showed similar results.

### DETAILED DESCRIPTION OF THE INVENTION

The present invention is based, at least in part, on the finding that human atheroma-associated endothelial cells (EC), smooth muscle cells (SMC) and macrophages express insterstitial collagenase MMP-8 in vitro, in response to proinflammatory cytokines, e.g., IL-1β, CD40L, TNFα, or LPS. MMP-8 colocalized with all three cell types within the atherosclerotic lesion in situ, particularly within sites of collagenolysis (i.e., vulnerable plaques). Since interstitial collagen, i.e., type I collagen, comprises one of the major load-bearing molecules within the plaque fibrous cap overlying the pro-coagulant lipid core, collagenolysis in advanced atherosclerotic lesions is believed to promote the evolution of rupture-prone lesions. These findings implicate MMP-8, the preferred substrate of which is type I collagen, in the pathogenic processes rendering atherosclerotic lesions prone to rupture. It is believed that dysregulation of collagen metabolism predisposes plaques to rupture. Rupture of atherosclerotic lesions triggers most acute clinical manifestations of atherosclerosis, such as myocardial infarction or stroke. Based on the discovery that MMP-8 is expressed in atheroma-associated cells, the present invention provides new modalities in the treatment and diagnosis of non-neutrophil-mediated inflammatory conditions, and in particular cardiovascular disorders, such as atherosclerosis. It has also been observed that MMP-8 is highly expressed in tissue samples obtained from patients suffering from chronic obstructive pulmonary disease (COPD) and inflammatory bowel disease (IBD), thus implicating MMP-8-mediated type I collagen degradation in these diseases, as well.

According to the present invention, MMP-8 represents a target for therapy and diagnosis of cardiovascular conditions. As described in greater detail below, inhibitors of the MMP-8 can be used to block or reduce the collagen proteolytic activity of this enzyme, thereby inhibiting collagen metabolism that predisposes plaques to rupture. In accordance with the present invention, MMP-8 inhibitors can be used in the treatment of non-neutrophil-mediated inflammatory conditions, and in particular cardiovascular disorders, such as atherosclerosis, myocardial infarction, aneurism, and stroke.

In order that the present invention may be more readily understood, certain terms are first defined. Additional definitions are set forth throughout the detailed description.

The term “MMP-8” refers to an interstitial collagenase which can initiate the breakdown of intact, triple-helical collagen. MMP-8, as well as other members of this MMP subfamily, catalyze the initial and rate-limiting cleavage of all three a-chains of type I, II, and III collagen at Gly⁷⁷⁵-Leu/Ile⁷⁷⁶, degrading the molecule into one-quarter and three-quarter fragments (Mitchell P G et al. J Clin Invest. 1996; 97: 76 1-8; Krane S M et al. J Biol Chem. 1996; 27 1: 28509-15). MMP-8 preferentially degrades type I collagen. The term MMP-8 preferably includes mammalian MMP-8 sequences, e.g., rodent, primate (e.g., monkey or human), but preferably human MMP-8 sequences. Human MMP-8, originally termed human “neutrophil-type collagenase” (“HNC”) or “collagenase-2”, was cloned from mRNA extracted from peripheral blood leukocytes of a patient with chronic granulocytic leukemia (Hasty K A et al. J Biol Chem. 1990; 265: 1142 1-4, the content of which are incorporated herein by reference). Also within this definition are variants thereof, as for example, differentially expressed variants, active or latent forms of MMP-8, MMP-8 polypeptides having conservative substitutions or non-essential amino acid substitutions, as well as MMP-8 polypeptide or nucleic acids having sequences substantially homologous to an MMP-8 sequence, preferably a human MMP-8 sequence.

The term “MMP” refers to a family of proteases (enzymes) involved in the degradation and remodeling of connective tissues. Members of this family of endopeptidase enzymes are secreted as proenzymes from various cell types that reside in or are associated with connective tissue, such as fibroblasts, monocytes, macrophages, endothelial cells, and invasive or metastatic tumor cells. MMP expression is stimulated by growth factors and cytokines in the local tissue environment, where these enzymes act to specifically degrade protein components of the extracellular matrix, such as collagen, proteoglycans (protein core), fibronectin and laminin. The MMP family members share a number of properties, including zinc and calcium dependence, secretion as zymogens, and 40-50% amino acid sequence homology. Exemplary MMP's in humans include three collagenases (interstitial collagenases), three stromelysins, two gelatinases, matrilysin, metalloelastase, and membrane-type MMP.

As used herein, the term “interstitial collagenases” refers to enzymes that catalyze the initial and rate-limiting cleavage of native collagen types I, II and III. Interstitial collagen fibrils resist degradation by most proteinases. The interstitial collagenases I (MMP-1), II (MMP-8), and III (MMP-13) are very specific matrix metalloproteases which can initiate the breakdown of intact, triple-helical collagen. The term “gelatinases” includes two distinct, but highly related, enzymes: a 72-kD enzyme (gelatinase A, HFG, MMP-2) secreted by fibroblasts and a wide variety of other cell types, and a 92-kD enzyme (gelatinase B, HNG, MMP-9) released by mononuclear phagocytes, neutrophils, corneal epithelial cells, tumor cells, cytotrophoblasts and keratinocytes. These gelatinases have been shown to degrade gelatins (denatured collagens), collagen types IV (basement membrane) and V, fibronectin and insoluble elastin.

The term “stromelysins” refers to members 1, 2 and 3, which have been shown to cleave a broad range of matrix substrates, including laminin, fibronectin, proteoglycans, and collagen types IV and IX in their non-helical domains.

Matrilysin (MMP-7, PUMP-1) has been shown to degrade a wide range of matrix substrates including proteoglycans, gelatins, fibronectin, elastin and laminin. Its expression has been documented in mononuclear phagocytes, rate uterine explants and sporadically in tumors. Other less characterized MMPs include macrophage metalloelastase (MME, MMP-12), membrane type MMP (MMP-14), and stromelysin-3 (MMP-11).

The term “atheroma” is intended to have its clinical meaning. It refers to a disease characterized by thickening and fatty degeneration of the inner coat of the arteries. “Atheroma-associated cells or tissues” refer to cells that localize to the vicinity of the atheroma, e.g., cells found at or near an atherosclerotic plaque or lesion. Such cells may be involved in pathological as well as non-pathological conditions. Examples of these cells include smooth muscle cells, endothelial cells and macrophages.

As used herein, the term “macrophage” refers to monocyte-derived cells that enter the extravascular pool and become resident in the tissues (i.e., they are the tissue form of monocytes). The term “macrophage” as used herein includes all cells from the monocyte/macrophage lineage, including mononuclear phagocytes (MNP's), monocytes, as well as specialized cells (e.g., atheroma-associated macrophages, alveolar macrophages, Kupffer cells, mesagial cells, microglial cells, and osteoclasts). Monocytes and macrophages have different morphology and size compared to neutrophils and lymphocytes, for example, these cells have a single nucleus and abundant granular cytoplasm. Monocytes form between 5 and 10% of the circulating white blood cells and have a short-half life, spending about 24 hours in the blood. Monocytes migrate in three ways: randomly, into the sites of inflammation, or in a tissue-directed way to become specialized cells. Several specialized forms of the mature cells exist, including alveolar macrophages in the lung, Kupffer cells in the liver, mesagial cells in the kidney, microglial cells in the brain, and osteoclast cells in the bone.

The term “cardiovascular disorders” or “disease” includes heart disorders, as well as disorders of the blood vessels of the circulation system caused by, e.g., abnormally high concentrations of lipids in the blood vessels.

As used herein, the term “atherosclerosis” is intended to have its clinical meaning. This term refers to a cardiovascular condition occurring as a result of lesion (e.g., plaque or streak) formation in the arterial walls. The formation of plaques or streaks results in a reduction in the size of the inner lining of the arteries. These plaques consist of foam cells filled with modified low-density lipoproteins, oxidized-LDL, decaying smooth muscle cells, fibrous tissue, clumps of blood platelets, cholesterol, and sometimes calcium. They tend to form in regions of disturbed blood flow and are found most often in people with high concentrations of cholesterol in the bloodstream. The number and thickness of plaques increase with age, causing loss of the smooth lining of the blood vessels and encouraging the formation of thrombi (blood clots). Sometimes fragments of thrombi break off and form emboli, which travel through the bloodstream and block smaller vessels. The thrombi or emboli can restrict the blood supply to the heart, brain, kidney and other organs eventually leading to end organ damage or death. The major causes of atherosclerosis are hypercholesterolemia, hypoalphoproteinemia, and hyperlipidemia marked by high circulating triglycerides in the blood. These lipids are deposited in the arterial walls, obstructing the blood flow and forming atherosclerotic plaques leading to death.

As used herein the term “hypercholesterolemia” is a condition with elevated levels of circulating total cholesterol, LDL-cholesterol and VLDL-cholesterol as per the guidelines of the Expert Panel Report of the National Cholesterol Educational Program (NCEP) of Detection, Evaluation of Treatment of high cholesterol in adults (see, Arch. Int. Med. (1988) 148, 36-39).

As used herein the term “hyperlipidemia” or “hyperlipemia” is a condition where the blood lipid parameters are elevated in the blood. This condition manifests an abnormally high concentration of fats. The lipid fractions in the circulating blood are, total cholesterol, low density lipoproteins, very low density lipoproteins and triglycerides.

As used herein the term “lipoprotein” such as VLDL, LDL and HDL, refers to a group of proteins found in the serum, plasma and lymph and are important for lipid transport. The chemical composition of each lipoprotein differs in that the HDL has a higher proportion of protein versus lipid, whereas the VLDL has a lower proportion of protein versus lipid.

As used herein, the term “triglyceride” means a lipid or neutral fat consisting of glycerol combined with three fatty acid molecules.

As used herein the term “xanthomatosis” is a disease evidenced by a yellowish swelling or plaques in the skin resulting from deposits of fat. The presence of xanthomas are usually accompanied by raised blood cholesterol levels.

As used herein the term “apolipoprotein B” or “apoprotein B” or “Apo B” refers to the protein component of the LDL cholesterol transport proteins. Cholesterol synthesized de novo is transported from the liver and intestine to peripheral tissues in the form of lipoproteins. Most of the apolipoprotein B is secreted into the circulatory system as VLDL.

As used herein the term “apolipoprotein A” or “apoprotein A” or “Apo A” refers to the protein component of the HDL cholesterol transport proteins.

“Procedural vascular trauma” includes the effects of surgical/medical-mechanical interventions into mammalian vasculature, but does not include vascular trauma due to the organic vascular pathologies listed hereinabove, or to unintended traumas, such as due to an accident. Thus, procedural vascular traumas within the scope of the present treatment method include (1) organ grafting or transplantation, such as transplantation and grafting of heart, kidney, liver and the like, e.g., involving vessel anastomosis; (2) vascular surgery, such as coronary bypass surgery, biopsy, heart valve replacement, atheroectomy, thrombectomy, and the like; (3) transcatheter vascular therapies (TVT) including angioplasty, e.g., laser angioplasty and PTCA procedures discussed hereinbelow, employing balloon catheters, or indwelling catheters; (4) vascular grafting using natural or synthetic materials, such as in saphenous vein coronary bypass grafts, dacron and venous grafts used for peripheral arterial reconstruction, etc.; (5) placement of a mechanical shunt, such as a PTFE hemodialysis shunt used for arteriovenous communications; and (6) placement of an intravascular stent, which may be metallic, plastic or a biodegradable polymer. See U.S. patent application Ser. No. 08/389,712, filed Feb. 15, 1995, which is incorporated by reference herein. For a general discussion of implantable devices and biomaterials from which they can be formed, see H. Kambic et al., “Biomaterials in Artificial Organs”, Chem. Eng. News, 30 (Apr. 14, 1986), the disclosure of which is incorporated by reference herein.

Small vessel disease includes, but is not limited to, vascular insufficiency in the limbs, peripheral neuropathy and retinopathy, e.g., diabetic retinopathy.

As used herein, an “endothelial cell disorder” includes a disorder characterized by aberrant, unregulated, or unwanted endothelial cell activity, e.g., proliferation, migration, angiogenesis, or vascularization; or aberrant expression of cell surface adhesion molecules or genes associated with angiogenesis, e.g., TIE-2, FLT and FLK. Endothelial cell disorders include tumorigenesis, tumor metastasis, psoriasis, diabetic retinopathy, endometriosis, Grave's disease, ischemic disease (e.g., atherosclerosis), and chronic inflammatory diseases (e.g., rheumatoid arthritis).

The term “agent” is used herein to denote a chemical compound, a mixture of chemical compounds, a biological macromolecule, a peptide, polypeptide (e.g., an antibody), small molecule, member of a combinatorial library, a peptide fragment, a peptidomimetic, or an extract made from biological materials such as bacteria, plants, fungi, or animal (particularly mammalian) cells or tissues. Agents can be evaluated for MMP-8 inhibitory activity by inclusion in screening assays described, for example, hereinbelow.

As used herein, an “MMP-8-specific inhibitor” refers to an inhibitor that (1) binds to MMP-8 with high affinity, e.g., an affinity of at least 1×10⁷ M⁻¹, preferably 1×10⁸ M⁻¹, 1×10⁹ M⁻¹, 1×10¹⁰ M⁻¹ or higher; (2) preferentially binds to MMP-8 with an affinity that is at least two-fold greater than its affinity for binding to other MMP's (e.g., MMP-1, MMP-2, MMP-3, MMP-9, or MMP-13); and (3) partially or completely blocks MMP-8 activity or expression. Examples of inhibitors include anti-MMP-8 antibodies and small molecule inhibitors.

As used herein, an “MMP-non-specific inhibitor” refers to an inhibitor that binds to at least two MMP's, or that binds an MMP other than MMP-8, and partially or completely blocks MMP activity or expression. For example, an MMP-non-specific inhibitor may bind to two or more MMP's chosen from e.g., MMP-1, MMP-2, MMP-3, MMP-8, MMP-9, or MMP-13.

As used herein, a “therapeutically effective amount” of an agent refers to an amount of an MMP-8 inhibitor which is effective, upon single or multiple dose administration to the subject, e.g., a patient, at inhibiting MMP-8 expression or activity, or in prolonging the survival of the subject with a non-neutrophil-mediated inflammatory disorder, cardiovascular or endothelial disorder, or disorder beyond that expected in the absence of such treatment.

As used herein, “inhibiting the expression or activity” of MMP-8 refers to a reduction, blockade of the expression or activity, e.g., collagenolysis (e.g., degradation of collagen I) and does not necessarily indicate a total elimination of the MMP-8 expression or activity.

As used herein, “a prophylactically effective amount” of an agent refers to an amount of an MMP-8 inhibitor which is effective, upon single- or multiple-dose administration to the patient, in preventing or delaying the occurrence of the onset or recurrence of a disorder as described herein.

The terms “induce”, “inhibit”, “potentiate”, “elevate”, “increase”, “decrease” or the like, e.g., which denote quantitative differences between two states, refer to at least statistically significant differences between the two states. For example, “an amount effective to inhibit the activity or expression of MMP-8 means that the level of activity or expression of MMP-8 in a treated sample will differ statistically significantly from the level of MMP-8 activity or expression in untreated cells. Such terms are applied herein to, for example, levels of expression, and levels activity.

As used herein, the term “substantially identical,” (or “substantially homologous”) is used herein to refer to a first amino acid that contains a sufficient number of identical or equivalent (e.g., with a similar side chain, e.g., conserved amino acid substitutions) amino acid residues to a second amino acid such that the first and second amino acid sequences have similar activities, e.g., the ability to degrade collagen (e.g., type I collagen).

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences have a common functional activity or encode a common domain or a common functional MMP-8 activity.

MMP-8 variants having sequences similar or homologous (e.g., at least about 85% sequence identity) to the sequences disclosed herein are also part of this application. In some embodiment, the sequence identity can be about 90%, 91%, 92%, 93%, 94%, 95%, 96%, 97%, 98%, 99% or higher. Alternatively, substantial identity exists when the nucleic acid segments will hybridize under selective hybridization conditions (e.g., highly stringent hybridization conditions), to the complement of the strand.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a Blossum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

As used herein, the term “hybridizes under stringent conditions” describes conditions for hybridization and washing. Stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. A preferred, example of stringent hybridization conditions are hybridization in 6×sodium chloride/sodium citrate (SSC) at about 45° C., followed by one or more washes in 0.2×SSC, 0.1% SDS at 50° C. Another example of stringent hybridization conditions are hybridization in 6×SSC at about 45° C., followed by one or more washes in 0.2×SSC, 0.1% SDS at 55° C. A further example of stringent hybridization conditions are hybridization in 6×SSC at about 45° C., followed by one or more washes in 0.2×SSC, 0.1% SDS at 60° C. Preferably, stringent hybridization conditions are hybridization in 6×SSC at about 45° C., followed by one or more washes in 0.2×SSC, 0.1% SDS at 65° C. Particularly preferred highly stringent conditions (and the conditions that should be used if the practitioner is uncertain about what conditions should be applied to determine if a molecule is within a hybridization limitation of the invention) are 0.5M sodium phosphate, 7% SDS at 65° C., followed by one or more washes at 0.2×SSC, 1% SDS at 65° C.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

A “non-essential” amino acid residue is a residue that can be altered from the wildtype sequence of a hybrid antibody, without abolishing or more preferably, without substantially altering a biological activity, whereas an “essential” amino acid residue results in such a change.

As used herein, the term “nucleic acid molecule” includes DNA molecules (e.g., a cDNA or genomic DNA) and RNA molecules (e.g., an mRNA) and analogs of the DNA or RNA generated, e.g., by the use of nucleotide analogs. The nucleic acid molecule can be single-stranded or double-stranded, but preferably is double-stranded DNA.

The term “isolated or purified nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

### Methods of Inhibiting MMP-8 Activity, Expression, or Processing

In one aspect, this invention features methods for inhibiting MMP-8 activity, expression, or processing by contacting MMP-8, MMP-8-expressing cells, or MMP-8 activators (e.g., upstream activators) with an agent that inhibits MMP-8 expression, activity, or processing. The method can be performed on cells in culture, e.g., in vitro or ex vivo, or can be performed on cells present in an animal subject, e.g., as part of an in vivo therapeutic or prophylactic protocol. The therapeutic regimen can be carried out on a human or other subject.

As used herein, the term “subject” is intended to include human and non-human animals. Non-limiting examples of human subjects include human patients suffering from a non-neutrophil-mediated inflammatory disorder, a cardiovascular or an endothelial disorder as described herein. The term “non-human animals” of the invention includes all vertebrates, e.g., mammals and non-mammals, such as non-human primates, rabbits, rodents (e.g., mice), sheep, dog, cow, chickens, amphibians, reptiles, etc.

The agents of the invention can be used to treat, and/or prevent disorders, such as non-neutrophil-mediated inflammatory disorder or a cardiovascular disorder.

Non-limiting examples of the non-neutrophil-mediated inflammatory disorders that can be treated or prevented include, but are not limited to, transplant rejection, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjögren's Syndrome, inflammatory bowel disease (IBD), Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, chronic obstructive pulmonary disease (COPD), interstitial lung fibrosis, graft-versus-host disease, and allergy such as, atopic allergy.

Preferred examples of cardiovascular disorders or diseases include e.g., atherosclerosis, aneurism, thrombosis, heart failure, ischemic heart disease, angina pectoris, myocardial infarction, sudden cardiac death, hypertensive heart disease; non-coronary vessel disease, such as arteriolosclerosis, small vessel disease, nephropathy, hypertriglyceridemia, hypercholesterolemia, hyperlipidemia, hypertension; or a cardiovascular condition associated with interventional procedures (“procedural vascular trauma”), such as restenosis following angioplasty, placement of a shunt, stent, synthetic or natural excision grafts, indwelling catheter, valve or other implantable devices.

Disorders involving the heart, include but are not limited to, heart failure, including but not limited to, cardiac hypertrophy, left-sided heart failure, and right-sided heart failure; ischemic heart disease, including but not limited to angina pectoris, myocardial infarction, chronic ischemic heart disease, aneurism, and sudden cardiac death; hypertensive heart disease, including but not limited to, systemic (left-sided) hypertensive heart disease and pulmonary (right-sided) hypertensive heart disease; valvular heart disease, including but not limited to, valvular degeneration caused by calcification, such as calcific aortic stenosis, calcification of a congenitally bicuspid aortic valve, and mitral annular calcification, and myxomatous degeneration of the mitral valve (mitral valve prolapse), rheumatic fever and rheumatic heart disease, infective endocarditis, and noninfected vegetations, such as nonbacterial thrombotic endocarditis and endocarditis of systemic lupus erythematosus (Libman-Sacks disease), carcinoid heart disease, and complications of artificial valves; myocardial disease, including but not limited to dilated cardiomyopathy, hypertrophic cardiomyopathy, restrictive cardiomyopathy, and myocarditis; pericardial disease, including but not limited to, pericardial effusion and hemopericardium and pericarditis, including acute pericarditis and healed pericarditis, and rheumatoid heart disease; neoplastic heart disease, including but not limited to, primary cardiac tumors, such as myxoma, lipoma, papillary fibroelastoma, rhabdomyoma, and sarcoma, and cardiac effects of noncardiac neoplasms; congenital heart disease, including but not limited to, left-to-right shunts—late cyanosis, such as atrial septal defect, ventricular septal defect, patent ductus arteriosus, and atrioventricular septal defect, right-to-left shunts—early cyanosis, such as tetralogy of fallot, transposition of great arteries, truncus arteriosus, tricuspid atresia, and total anomalous pulmonary venous connection, obstructive congenital anomalies, such as coarctation of aorta, pulmonary stenosis and atresia, and aortic stenosis and atresia, asthma, emphysema and chronic pulmonary disease and disorders involving cardiac transplantation.

Disorders involving blood vessels include, but are not limited to, responses of vascular cell walls to injury, such as endothelial dysfunction and endothelial activation and intimal thickening; vascular diseases including, but not limited to, congenital anomalies, such as arteriovenous fistula, atherosclerosis, and hypertensive vascular disease, such as hypertension; inflammatory disease—the vasculitides, such as giant cell (temporal) arteritis, Takayasu arteritis, polyarteritis nodosa (classic), Kawasaki syndrome (mucocutaneous lymph node syndrome), microscopic polyanglitis (microscopic polyarteritis, hypersensitivity or leukocytoclastic anglitis), Wegener granulomatosis, thromboanglitis obliterans (Buerger disease), vasculitis associated with other disorders, and infectious arteritis; Raynaud disease; aneurisms and dissection, such as abdominal aortic aneurisms, syphilitic (luetic) aneurisms, and aortic dissection (dissecting hematoma); disorders of veins and lymphatics, such as varicose veins, thrombophlebitis and phlebothrombosis, obstruction of superior vena cava (superior vena cava syndrome), obstruction of inferior vena cava (inferior vena cava syndrome), and lymphangitis and lymphedema; tumors, including benign tumors and tumor-like conditions, such as hemangioma, lymphangioma, glomus tumor (glomangioma), vascular ectasias, and bacillary angiomatosis, and intermediate-grade (borderline low-grade malignant) tumors, such as Kaposi sarcoma and hemangloendothelioma, and malignant tumors, such as angiosarcoma and hemangiopericytoma; and pathology of therapeutic interventions in vascular disease, such as balloon angioplasty and related techniques and vascular replacement, such as coronary artery bypass graft surgery.

Endothelial cell disorders include, but are not limited to cancers, tumorigenesis, tumor metastasis, psoriasis, diabetic retinopathy, endometriosis, Grave's disease, ischemic disease (e.g., atherosclerosis), and chronic inflammatory diseases (e.g., rheumatoid arthritis).

Examples of cancers include carcinomas, sarcomas, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

In some embodiments, the therapeutic and prophylactic uses of the agents of the invention, further include the administration of a second agent, e.g., a non-specific MMP inhibitor, a cholesterol lowering agent, or an interventional as a combination therapies. The term “in combination” in this context means that the agents, or agent and procedures are given substantially contemporaneously, either simultaneously or sequentially. If given sequentially, at the onset of administration of the second agent or procedure, the first agent is preferably still detectable at effective concentrations at the site of treatment. For example, the combination therapy can include an agent of the present invention coformulated with, and/or coadministered with, one or more additional therapeutic agents, e.g., one or more MMP inhibitors, cytotoxic or cytostatic agents and/or immunosuppressants. For example, the agents of the invention or antibody binding fragments thereof may be coformulated with, and/or coadministered with, one or more additional MMP inhibitors.

The agents of the invention may be administered in combination with lipid lowering agents. Current combination therapy therapies using combinations of niacin and statins are being used with positive results to treat hyperlipidemia (Guyton, J R. (1999) Curr Cardiol Rep. 1(3):244-250; Otto, C. et al. (1999) Internist (Berl) 40(12):1338-45). Other useful drug combinations include those derived by addition of fish oil, bile acid binding resins, or stanol esters, as well as nonstatin combinations such as niacin-resin or fibrate-niacin (Guyton, J R. (1999) supra). For examples of dosages and administration schedules of the cholesterol lowering agents, the teachings of Guyton, J R. (1999) supra, Otto, C. et al. (1999) supra, Guyton, J R et al. (1998) Am J Cardiol 82(12A):82U-86U; Guyton, J R et al. (1998) Am J Cardiol. 82(6):737-43; Vega, G L et al. (1998) Am J. Cardiol. 81(4A):36B-42B; Schectman, G. (1996) Ann Intern Med. 125(12):990-1000; Nakamura, H. et al. (1993) Nippon Rinsho 51(8):2101-7; Goldberg, A. et al. (2000) Am J Cardiol 85(9):1100-5; Morgan, J M et al. (1996) J Cardiovasc. Pharmac. Ther. 1(3):195-202; Stein, E A et al. (1996) J Cardiovasc Pharmacol Ther 1(2):107-116; and Goldberg, A C (1998) Am J Cardiol 82(12A):35U-41U, are expressly incorporated by reference.

As used herein, “cholesterol lowering agents” include agents which are useful for lowering serum cholesterol such as for example bile acid sequestering resins (e.g. colestipol hydrochloride or cholestyramine), fish oil, stanol esters, an ApoAII-lowering agent, a VLDL lowering agent, an ApoAI-stimulating agent, fibric acid derivatives (e.g. clofibrate, fenofibrate, or gemfibrozil), thiazolidenediones (e.g. troglitazone, pioglitazone, ciglitazone, englitazone, rosiglitazone), or HMG-CoA reductase inhibitors (e.g. statins, such as fluvastatin sodium, lovastatin, pravastatin sodium, simvastatin, atorvastatin calcium, cerivastatin), as well as nicotinic acid, niacin, or probucol.

“VLDL-lowering agent” includes an agent which decreases the hepatic synthesis of triglyceride-rich lipoproteins or increases the catabolism of triglyceride-rich lipoproteins, e.g., fibrates such as gemfibrozil, or the statins, increases the expression of the apoE-mediated clearance pathway, or improves insulin sensitivity in diabetics, e.g., the thiazolidene diones.

### Methods of Identifying MMP-8 Specific Inhibitors

In another aspect, the invention features methods for screening for an agent that inhibits the activity or expression of MMP-8. Such polypeptides can be assayed for their ability to bind, or to inhibit the enzyme activity (e.g., collagen or a fluorogenic peptide substrate degradation). Fluorogenic MMP peptide substrates are known in the art and are commercially available from e.g., Chondrex or Chemicon (Oncogene).

MMP-8 can be purified, e.g., by fusing a nucleic acid encoding the polypeptide to an affinity tag (e.g., an epitope tag such as Flag, HA, or myc, glutathione-S-transferase, chitin binding protein, maltose binding protein, or dihydrofolate reductase) See Kolodziej and Young (1991) Methods Enz. 194:508-519 for general methods of providing an epitope tag. Alternatively, the polypeptide can be purified using standard purification techniques, such as immunoaffinity chromatography, ammonium sulfate precipitation, ion exchange chromatography, filtration, electrophoresis, hydrophobic interaction chromotography, and others.

The production of MMP-8 specific inhibitors is described in more detail below.

### Anti-MMP-8 Antibodies

Antibodies are useful reagents for many embodiments of the invention. An antibody against MMP-8, e.g., human MMP-8 can be used as 1) a reagent to detect the presence of MMP-8 (for example, in a diagnostic assay) or 2) a reagent to alter the activity or function of MMP-8.

An antibody can be an antibody or a fragment thereof, e.g., an antigen binding portion thereof. As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4.

The antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

The term “antigen-binding fragment” of an antibody (or simply “antibody portion,” or “fragment”), as used herein, refers to one or more fragments of a full-length antibody that retain the ability to specifically bind to an antigen (e.g., a polypeptide encoded by an atherosclerosis (MMP-8)-associated nucleic acid). Examples of binding fragments encompassed within the term “antigen-binding fragment” of an antibody include (i) a Fab fragment, a monovalent fragment consisting of the VL, VH, CL and CHI domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate nucleic acids, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies.

The terms “monoclonal antibody” or “monoclonal antibody composition” as used herein refer to a preparation of antibody molecules of single molecular composition. A monoclonal antibody composition displays a single binding specificity and affinity for a particular epitope.

The antibodies described herein can be human, rodent, humanized, or chimeric antibodies.

Methods of producting antibodies are well known in the art. For example, a monoclonal antibody against a target (e.g., a polypeptide encoded by an atherosclerosis (MMP-8)-associated nucleic acid) can be produced by a variety of techniques, including conventional monoclonal antibody methodology e.g., the standard somatic cell hybridization technique of Kohler and Milstein (1975) Nature 256: 495. Although somatic cell hybridization procedures are preferred, in principle, other techniques for producing monoclonal antibody can be employed e.g., viral or oncogenic transformation of B lymphocytes. The preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

For example, antibodies to a polypeptide encoded by an atherosclerosis (MMP-8)-associated nucleic acid can be raised, e.g., by immunization of rabbits with purified polypeptide or with peptides obtained by conventional methods of chemical synthesis, e.g., Merrifield solid phase synthesis. The antisera or monoclonal antibodies can be tested to determine whether they show the ability to discriminate between the polypeptide and other antigens, e.g., by dot immunoblotting or by ELISA. To select a high-affinity reagent with low background signal in the high-throughput screening assay, the candidate antiserum or monoclonal antibody can be further tested under the conditions to be used in the high-throughput screening assay.

Human monoclonal antibodies (mAbs) directed against human proteins can be generated using transgenic mice whose genomes include the human immunoglobulin loci instead of the murine loci. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

Monoclonal antibodies can also be generated by other methods known to those skilled in the art of recombinant DNA technology. An alternative method, referred to as the “combinatorial antibody display” method, has been developed to identify and isolate antibody fragments having a particular antigen specificity, and can be utilized to produce monoclonal antibodies (for descriptions of combinatorial antibody display see e.g., Sastry et al. 1989 PNAS 86:5728; Huse et al. 1989 Science 246:1275; and Orlandi et al. 1989 PNAS 86:3833). After immunizing an animal with an immunogen as described above, the antibody repertoire of the resulting B-cell pool is cloned. Methods are generally known for obtaining the DNA sequence of the variable regions of a diverse population of immunoglobulin molecules by using a mixture of oligomer primers and PCR. For instance, mixed oligonucleotide primers corresponding to the 5′ leader (signal peptide) sequences and/or framework 1 (FR1) sequences, as well as primer to a conserved 3′ constant region primer can be used for PCR amplification of the heavy and light chain variable regions from a number of murine antibodies (Larrick et al., 1991, Biotechniques 11:152-156). A similar strategy can also been used to amplify human heavy and light chain variable regions from human antibodies (Larrick et al., 1991, Methods: Companion to Methods in Enzymology 2:106-110).

The amplified fragments can be expressed by a population of display packages, preferably derived from filamentous phage, to form an antibody display library. Ideally, the display package comprises a system that allows the sampling of very large variegated antibody display libraries, rapid sorting after each affinity separation round, and easy isolation of the antibody nucleic acid from purified display packages. In addition to commercially available kits for generating phage display libraries (e.g., the Pharmacia Recombinant Phage Antibody System, catalog no. 27-9400-01; and the Stratagene SurfZAP™ phage display kit, catalog no. 240612), examples of methods and reagents particularly amenable for use in generating a variegated antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982. The fragments can also be variegated prior to expression, e.g., by random or directed mutagenesis or by DNA shuffling (Maxygen, CA).

Once displayed on the surface of a display package (e.g., filamentous phage), the antibody library is screened with the target antigen, or peptide fragment thereof, to identify and isolate packages that express an antibody having specificity for the target antigen. Nucleic acid encoding the selected antibody can be recovered from the display package (e.g., from the phage genome) and subcloned into other expression vectors by standard recombinant DNA techniques.

In certain embodiments, the V region domains of heavy and light chains can be expressed on the same polypeptide, joined by a flexible linker to form a single-chain Fv fragment, and the scFV nucleic acid subsequently cloned into the desired expression vector or phage genome. As generally described in MeCafferty et al., Nature (1990) 348:552-554, complete V_{H} and V_{L} domains of an antibody, joined by a flexible (Gly₄-Ser)₃ linker can be used to produce a single chain antibody which can render the display package separable based on antigen affinity. Isolated scFV antibodies immunoreactive with the antigen can subsequently be formulated into a pharmaceutical preparation for use in the subject method.

The Fv binding surface of a particular antibody molecule can be further engineered, e.g., on the basis of sequence data for V_{H} and V_{L} (the latter of which may be of the κ or λ chain type). Details of the protein surface that comprises the binding determinants can be obtained from antibody sequence information, by a modeling procedure using previously determined three-dimensional structures from other antibodies obtained from NMR studies or crytallographic data. See for example Bajorath, J. and S. Sheriff, 1996, Proteins: Struct., Funct., and Genet. 24 (2), 152-157; Webster, D. M. and A. R. Rees, 1995, “Molecular modeling of antibody-combining sites,” in S. Paul, Ed., Methods in Molecular Biol. 51, Antibody Engineering Protocols, Humana Press, Totowa, N.J., pp 17-49; and Johnson, G., Wu, T. T. and E. A. Kabat, 1995, “Seqhunt: A program to screen aligned nucleotide and amino acid sequences,” in Methods in Molecular Biol. 51, op. cit., pp 1-15. Protein engineering by molecular modeling is one method for producing a modified antibody.

The term “modified antibody” is also intended to include antibodies, such as monoclonal antibodies, chimeric antibodies, and humanized antibodies which have been modified by, e.g., deleting, adding, or substituting portions of the antibody. For example, an antibody can be modified by deleting the hinge region, thus generating a monovalent antibody. Any modification is within the scope of the invention so long as the antibody has at least one antigen binding region specific.

Chimeric mouse-human monoclonal antibodies (i.e., chimeric antibodies) can be produced by recombinant DNA techniques known in the art. For example, a nucleic acid encoding the Fc constant region of a murine (or other species) monoclonal antibody molecule is digested with restriction enzymes to remove the region encoding the murine Fc, and the equivalent portion of a nucleic acid encoding a human Fc constant region is substituted. (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

The chimeric antibody can be further humanized by replacing sequences of the Fv variable region which are not directly involved in antigen binding with equivalent sequences from human Fv variable regions. General reviews of humanized chimeric antibodies are provided by Morrison, S. L., 1985, Science 229:1202-1207 and by Oi et al., 1986, BioTechniques 4:214. Those methods include isolating, manipulating, and expressing the nucleic acid sequences that encode all or part of immunoglobulin Fv variable regions from at least one of a heavy or light chain. The recombinant DNA encoding the chimeric antibody, or fragment thereof, can then be cloned into an appropriate expression vector. Suitable humanized antibodies can alternatively be produced by CDR substitution U.S. Pat. No. 5,225,539; Jones et al. 1986 Nature 321:552-525; Verhoeyan et al. 1988 Science 239:1534; and Beidler et al. 1988 J. Immunol. 141:4053-4060.

All of the CDRs of a particular human antibody may be replaced with at least a portion of a non-human CDR or only some of the CDRs may be replaced with non-human CDRs. It is only necessary to replace the number of CDRs required for binding of the humanized antibody to MMP-8.

An antibody can be humanized by any method, which is capable of replacing at least a portion of a CDR of a human antibody with a CDR derived from a non-human antibody. Winter describes a method that can be used to prepare the humanized antibodies of the present invention (UK Patent Application GB 2188638A, filed on Mar. 26, 1987). The human CDRs may be replaced with non-human CDRs using oligonucleotide site-directed mutagenesis.

Also within the scope of the invention are chimeric and humanized antibodies in which specific amino acids have been substituted, deleted or added. In particular, preferred humanized antibodies have amino acid substitutions in the framework region, such as to improve binding to the antigen. For example, in a humanized antibody having mouse CDRs, amino acids located in the human framework region can be replaced with the amino acids located at the corresponding positions in the mouse antibody. Such substitutions are known to improve binding of humanized antibodies to the antigen in some instances.

An antibody or antibody portion of the invention can be derivatized or linked to another functional molecule (e.g., another peptide or protein). Accordingly, the antibodies and antibody portions of the invention are intended to include derivatized and otherwise modified forms of the anti-MMP-8 antibodies described herein, including immunoadhesion molecules. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate associate of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an anti-MMP-8 antibody or antibody portion of the invention may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with biotin, and detected through indirect measurement of avidin or streptavidin binding.

### Design of Chemical MMP-8 Inhibitors/Small Molecule Inhibitors

The design and uses of MMP inhibitors are reviewed, for example, in J. Enzyme Inhibition, 2, 1-22 (1987); Progress in Medicinal Chemistry 29, 271-334 (1992); Current Medicinal Chemistry, 2, 743-762 (1995); Exp. Opin. Ther. Patents, 5, 1287-1296 (1995); and Drug Discovery Today, 1, 16-26 (1996), the contents of all of which are hereby incorporated by reference. MMP inhibitors are also the subject of numerous patents and patent applications. In the majority of these publications, the preferred inventive compounds are hydroxamic acids, as it has been well-established that the hydroxamate function is the optimal zinc-coordinating functionality for binding to the active site of MMPs. For example, the hydroxamate inhibitors described in the literature are generally 100 to 1000-fold more potent than the corresponding inhibitors wherein the hydroxamic acid functionality is replaced by a carboxylic acid functionality. Nevertheless, hydroxamic acids tend to exhibit relatively poor bioavailability. Other preferred inhibitors are carboxylic acid inhibitors that possess inhibitory potency against certain of the MMPs that is comparable to the potency of the hydroxamic acid inhibitors that have been reported in the literature. The following patents and patent applications disclose carboxylic acid inhibitors that are monoamine derivatives of substituted succinic acids: Celltech Ltd.: EP-A-0489577 (WO 92/099565), EP-A-0489579, WO 93/24475, WO 93/244449; British Biotech Pharameuticals Ltd.: WO 95/32944, WO 95/19961; Sterling Winthrop, Inc.: U.S. Pat. No. 5,256,657; Sanofi Winthrop, Inc.: WO 95/22966; and Syntex (U.S.A.) Inc. WO 94/04735, WO 95/12603, and WO 96/16027.

Several groups have reported the synthesis and design of MMP-8 specific inhibitors. For example, the synthesis of malonic acid-based MMP-8 inhibitors is described in Graf von Roedern et al. (1998) J. Med. Chem. 41(3):339-345. The synthesis of bis-substituted malonic acid hydroxamate MMP-8 inhibitors is described in Graf von Roedern et al. (1998) J. Med. Chem. 41(16):3041-3047. The crystal structure of human MMP-8 complexed with a primed or unprimed inhibitor is known in the art (see Gavuzzo, E. et al. (2000) J. Med. Chem. 43(18):3377-3385). Based on such structural information, the design of combined inhibitors assembled to interact with both primed and unprimed regions of the MMP-8 active cleft can be carried out. The contents of all of these references are hereby incorporated by reference.

Designed inhibitors, or “test agents” or “test compounds” can be tested by assessing binding to MMP-8 (e.g., using surface plasmon resonance, NMR, or spectroscopy), and/or enzymatic activity. The activity of the agents as inhibitors of MMP-8 activity may be measured by any of the methods available to those skilled in the art, including in vivo and in vitro assays. Examples of suitable assays for activity measurements include the fluorometric determination of the hydrolysis rate of a fluorescently-labeled peptide substrate, which is described herein. Fluorogenic MMP peptide substrates are known in the art and are commercially available from e.g., Chondrex or Chemicon (Oncogene).

Libraries of compounds can also be tested. A test compound can be a large or small molecule, for example, an organic compound with a molecular weight of about 100 to 10,000; 200 to 5,000; 200 to 2000; or 200 to 1,000 daltons. A test compound can be any chemical compound, for example, a small organic molecule, a polypeptide, a nucleic acid, or a peptide nucleic acid. Small molecules include, but are not limited to, metabolites, metabolic analogues, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds). Compounds and components for synthesis of compounds can be obtained from a commercial chemical supplier, e.g., Sigma-Aldrich Corp. (St. Louis, Mo.). The test compound or compounds can be naturally occurring, synthetic, or both. A test compound can be the only substance assayed by the method described herein. Alternatively, a collection of test compounds can be assayed either consecutively or concurrently by the methods described herein.

A high-throughput method can be used to screen large libraries of chemicals. Such libraries of candidate compounds can be generated or purchased e.g., from Chembridge Corp. (San Diego, Calif.). Libraries can be designed to cover a diverse range of compounds. For example, a library can include 10,000, 50,000, or 100,000 or more unique compounds. Merely by way of illustration, a library can be constructed from heterocycles including pyridines, indoles, quinolines, furans, pyrimidines, triazines, pyrroles, imidazoles, naphthalenes, benzimidazoles, piperidines, pyrazoles, benzoxazoles, pyrrolidines, thiphenes, thiazoles, benzothiazoles, and morpholines. Alternatively, a class or category of compounds can be selected to mimic the chemical structures of malate, oxaloacetate, amocarzine and suramin. A library can be designed and synthesized to cover such classes of chemicals, e.g., as described in DeWitt et al., (1993) Proc. Natl. Acad. Sci. U.S.A. 90:6909; Erb et al., (1994) Proc. Natl. Acad. Sci. USA 91:11422; Zuckermann et al. (1994). J. Med. Chem. 37:2678; Cho et al., (1993) Science 261:1303; Carrell et al., (1994) Angew. Chem. Int. Ed. Engl. 33:2059; Carell et al., (1994) Angew. Chem. Int. Ed. Engl. 33:2061; and in Gallop et al., (1994) J. Med. Chem. 37:1233.

In addition, libraries of compounds of the invention can be prepared according to a variety of methods, some of which are known in the art. For example, a “split-pool” strategy can be implemented in the following way: beads of a functionalized polymeric support are placed in a plurality of reaction vessels; a variety of polymeric supports suitable for solidphase peptide synthesis are known, and some are commercially available (for example, see, e.g., M. Bodansky “Principles of Peptide Synthesis”, 2nd edition, Springer-Verlag, Berlin (1993)). To each aliquot of beads is added a solution of a different activated amino acid, and the reactions are allow to proceed to yield a plurality of immobilized amino acids, one in each reaction vessel. The aliquots of derivatized beads are then washed, “pooled” (i.e., recombined), and the pool of beads is again divided, with each aliquot being placed in a separate reaction vessel. Another activated amino acid is then added to each aliquot of beads. The cycle of synthesis is repeated until a desired peptide length is obtained. The amino acid residues added at each synthesis cycle can be randomly selected; alternatively, amino acids can be selected to provide a “biased” library, e.g., a library in which certain portions of the inhibitor are selected non-randomly, e.g., to provide an inhibitor having known structural similarity or homology to a known peptide capable of interacting with an antibody, e.g., the an anti-idiotypic antibody antigen binding site. It will be appreciated that a wide variety of peptidic, peptidomimetic, or non-peptidic compounds can be readily generated in this way.

The “split-pool” strategy results in a library of peptides, e.g., inhibitors, which can be used to prepare a library of test compounds of the invention. In another illustrative synthesis, a “diversomer library” is created by the method of Hobbs DeWitt et al. (Proc. Natl. Acad. Sci. U.S.A. 90:6909 (1993)). Other synthesis methods, including the “tea-bag” technique of Houghten (see, e.g., Houghten et al., Nature 354:84-86 (1991)) can also be used to synthesize libraries of compounds according to the subject invention.

Libraries of compounds can be screened to determine whether any members of the library have a desired activity, and, if so, to identify the active species. Methods of screening combinatorial libraries have been described (see, e.g., Gordon et al., J Med. Chem., supra). Soluble compound libraries can be screened by affinity chromatography with an appropriate receptor to isolate ligands for a polypeptide encoded by an atherosclerosis (MMP-8)associated nucleic acid, followed by identification of the isolated ligands by conventional techniques (e.g., mass spectrometry, NMR, and the like). Immobilized compounds can be screened by contacting the compounds with a polypeptide encoded by an atherosclerosis (MMP-8)-associated nucleic acid; preferably, the polypeptide is conjugated to a label (e.g., fluorophores, colorimetric enzymes, radioisotopes, luminescent compounds, and the like) that can be detected to indicate binding. Alternatively, immobilized compounds can be selectively released and allowed to diffuse through a membrane to interact with a polypeptide. Exemplary assays useful for screening the libraries of the invention are described below.

In still another embodiment, large numbers of test compounds can be simultaneously tested for binding activity. For example, test compounds can be synthesized on solid resin beads in a “one bead-one compound” synthesis; the compounds can be immobilized on the resin support through a photolabile linker. A plurality of beads (e.g., as many as 100,000 beads or more) can then be combined with yeast cells and sprayed into a plurality of “nano-droplets”, in which each droplet includes a single bead (and, therefore, a single test compound). Exposure of the nano-droplets to UV light then results in cleavage of the compounds from the beads. It will be appreciated that this assay format allows the screening of large libraries of test compounds in a rapid format.

Combinatorial libraries of compounds can be synthesized with “tags” to encode the identity of each member of the library (see, e.g., W. C. Still et al., U.S. Pat. No. 5,565,324 and PCT Publication Nos. WO 94/08051 and WO 95/28640). In general, this method features the use of inert, but readily detectable, tags, that are attached to the solid support or to the compounds. When an active compound is detected (e.g., by one of the techniques described above), the identity of the compound is determined by identification of the unique accompanying tag. This tagging method permits the synthesis of large libraries of compounds which can be identified at very low levels. Such a tagging scheme can be useful, e.g., in the “nano-droplet” screening assay described above, to identify compounds released from the beads.

In preferred embodiments, the libraries of transcriptional modulator compounds of the invention contain at least 30 compounds, more preferably at least 100 compounds, and still more preferably at least 500 compounds. In preferred embodiments, the libraries of transcriptional modulator compounds of the invention contain fewer than 10⁹ compounds, more preferably fewer than 10⁸ compounds, and still more preferably fewer than 10⁷ compounds.

### Double-stranded RNA, Antisense RNA, and Ribozyme Inhibitors

Also featured are double-stranded RNA, antisense RNA, and ribozyme inhibitors of MMP-8. A double-stranded RNA (dsRNA) molecule includes a sequence that, typically, is a fragment of a mRNA molecule, which is hybridized to a complementary strand of RNA. dsRNA that is homologous to a sequence in an expressed gene (e.g., an mRNA) has been found, in many cases, to inhibit the transcription or translation of the corresponding mRNA. This is true even when the injected dsRNA is present at levels far below the levels of the corresponding RNA. This was first found in C. elegans, where dsRNA can be injected or even fed to the worms and thereby lead to “inactivation” of the corresponding gene. Thus, there is a mechanism by which dsRNA is capable of crossing the cell membrane. It has also been found recently that small RNAs (that form hairpins) are expressed in worms and other organisms that regulate gene actity by this mechanism. It was subsequently found that dsRNA could lead to gene inactivation in flies and human cells, as well. In human cells, however, there is a narrow window of size for the dsRNA to be effective. Specifically, dsRNA of about 21 bps is most effective. See Harborth et al. (2001), J Cell Sci. 114(24):4557-65, the contents of which are incorporated herein by reference.

An “antisense” nucleic acid includes a sequence that is complementary to the coding strand of a nucleic acid of the nucleic acid. The complementarity can be in a coding region of the coding strand or in a noncoding region, e.g., a 5′ or 3′ untranslated region, e.g., the translation start site. The antisense nucleic acid can be produced from a cellular promoter (e.g., a RNA polymerase II or III promoter), or can be introduced into a cell, e.g., using a liposome. For example, the antisense nucleic acid can be a synthetic oligonucleotide having a length of about 10, 15, 20, 30, 40, 50, 75, 90, 120 or more nucleotides in length.

An antisense nucleic acid can be synthesized chemically or produced using enzymatic reagents, e.g., a ligase. An antisense nucleic acid can also incorporate modified nucleotides, and artificial backbone structures, e.g., phosphorothioate derivative, and acridine substituted nucleotides.

The antisense nucleic acid can be a ribozyme. The ribozyme can be designed for to specifically cleave RNA, e.g., an mRNA for the nucleic acid. Methods for designing such ribozymes are described in U.S. Pat. No. 5,093,246 or Haselhoff and Gerlach (1988) Nature 334:585-591. For example, the ribozyme can be a derivative of Tetrahymena L-19 IVS RNA in which the nucleotide sequence of the active site is modified to be complementary to a target region of the nucleic acid (see, e.g., Cech et al. U.S. Pat. No. 4,987,071; and Cech et al. U.S. Pat. No. 5,116,742).

An antisense agent directed against a nucleic acid can be a peptide nucleic acid (PNA). See Hyrup B. et al (1996) Bioorganic & Medicinal Chemistry 4: 5-23) for methods and a description of the replacement of the deoxyribose phosphate backbone for a pseudopeptide backbone. A PNA can specifically hybridize to DNA and RNA under conditions of low ionic strength as a result of its electrostatic properties. The synthesis of PNA oligomers can be performed using standard solid phase peptide synthesis protocols as described in Hyrup B. et al (1996) supra; Perry-O'Keefe et al. Proc. Natl. Acad. Sci. 93:14670-675.

### Pharmaceutical Compositions

The present invention is further directed to methods of inhibiting matrix metalloproteinase activity that comprise contacting the protease with an effective amount of an agent as described herein, or a pharmaceutically acceptable prodrug or a pharmaceutically acceptable salt or solvate thereof. For example, one can inhibit matrix metalloproteinase activity in mammalian tissue by administering an agent or a pharmaceutically acceptable prodrug or a pharmaceutically acceptable salt or solvate thereof A composition containing an effective amount of an agent identified as described herein can be administered to a subject requiring treatment, e.g., for a non-neutrophil-mediated inflammatory, or a cardiovascular or endothelial cell disorder.

The composition can be administered parenterally, intravenously, topically, orally, buccally, nasally, rectally, subcutaneously, intramuscularly, or intraperitoneally. In one implementation, the composition is injected, e.g., into a vein.

The composition of the treatment is formulated to be compatible with the route of administration. The composition can formulated as a tablet, capsule, solution, or powder.

A solution for parenteral, intradermal, or subcutaneous administration can include: a sterile diluent such as water, saline, glycerine, fixed oils, polyethylene glycols, propylene glycol, or other synthetic solvents; an antibacterial agents such as benzyl alcohol or methyl parabens; an antioxidant such as ascorbic acid or sodium bisulfite; a chelating agent; a buffering agent such as acetate or phosphate. The solution can be stored in ampoules, disposable syringes, or plastic or glass vials.

A formulation for injection or intravenous administration can include a carrier which is a solvent or a dispersion medium. Suitable carriers include such water, physiological saline, bacteriostatic water, Cremophor EL™ (BASF, Parsippany, N.J.) phosphate buffered saline (PBS), ethanol, polyols (e.g., glycerol, glycol, propylene glycol, and the like), and mixtures thereof. These compositions must be sterile and fluid to allow injection. Fluidity can be maintained with a coating such as lecithin or a surfactant. Microbial contamination can prevented by the inclusion of antibacterial and antifungal agents, such as parabens, chlorobutanol, phenol, ascorbic acid, and thimerosal. Sugars and polyalcohols, such as manitol, sorbitol, sodium chloride, can be used to maintain isotonicity in the composition.

Sterility can be insured by filter sterilization of the solution. Alternatively, the solution can be produced from components that were individually filter sterilized. A filter-sterilized component can be vacuum dried or freeze dried to produce a sterile powder. Such a powder can be rehydrated prior to injection with a sterile carrier solution.

Oral compositions include tablets, capsules, troches, suspensions, and solutions. Such compositions can be fashioned with an inert diluent or an edible carrier. Capsules are made by combining an appropriate diluent with the compound and filling the capsule with the mixture. Common diluents are starches such as powdered cellulose, or sugars such as sucrose, fructose, or mannitol. Tablets are made by wet or dry granulation or by compression. In addition to the desired compound, compositions for tablets can include: a binder such as microcrystalline cellulose, or gelatin; an excipient such as a starch, a sugar (e.g., lactose, fructose, glucose, methylcellulose, ethylcellulose), a gum (e.g. gum tragacanth, acacia); a disintegrating agent(e.g., alginic acid, Primogel, or corn starch); a lubricant (e.g., magnesium stearate or Sterotes); a glidant (e.g., colloidal silicon dioxide); a sweetening agent (e.g., sucrose or saccharin); a flavoring agent (e.g., peppermint, methyl salicylate, or orange flavoring); or any compound of a similar nature. Biodegradable polymers such as poly-D,L-lactide-co-glycolide or polyglycolide, can be used as a matrix to delay the release of the composition (see e.g., U.S. Pat. Nos. 5,417,986, 4,675,381, and 4,450,150).

Therapeutic compositions can be administered with medical devices known in the art. For example, in a preferred embodiment, a therapeutic composition of the invention can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

### Dosage

An appropriate dosage of the agent for treatment can be determined. An effective amount of the agent can be an amount required to alleviate a symptom or an amount required to alter a nucleic acid expression profile of a sample from the subject, e.g., so that it is more similar to a desired . Determination of the amount or dose required to treat an individual subject is routine to one skilled in the art, e.g., a physician, pharmacist, or researcher. First, the toxicity and therapeutic efficacy of the compound is determined. Routine protocols are available for determining the LD₅₀ (the dose lethal to 50% of the population) and the ED₅₀ (the dose therapeutically effective in 50% of the population) in non-human animals. The therapeutic index is measured as the ratio of the LD₅₀/ED₅₀. Compounds, formulations, and methods of administration with high therapeutic indices are preferable as such treatments have little toxicity at dosages which provide high efficacy.

In formulating a dosage range for use in humans, the effective dose of the agent can be estimated from studies with test cells or an experimental animal. For example, therapeutically effective dosages in a cell culture assays can be about 0.1 ng/ml, 50 ng/ml, 500 ng/ml, 5 μg/ml, and 500 μg/ml of the agent. A dose can be formulated in an animal in order to achieve a circulating plasma concentration of the agent that falls in this range. An exemplary dose produces a plasma concentration which exceeds the ED₅₀ (i.e., the concentration of the test compound which achieves a half-maximal inhibition of symptoms) as determined in cell culture assays or in an experimental animal. The circulating plasma concentration can be determined, for example, by obtaining a blood sample, and by analyzing the sample with high performance liquid chromatography or mass spectroscopy.

### Diagnostic Methods

In yet another aspect, detection of MMP-8 RNA and/or protein expression can provide a useful diagnostic, prognostic method for detecting, or staging a non-neutrophil-mediated inflammatory disorder or a cardiovascular or endothelial disorder. For example, as described in the appended examples, we have observed that MMP-8 is selectively expressed in atheroma-associated cells. Thus, MMP-8 appears to be a sensitive marker for distinguishing disorders involving those cells. The amount of specific MMP-8 RNA or protein may be measured using any method known to those of skill in the art to be suitable. For example, RNA expression may be detected using Northern blots or RNA-based polymerase chain reaction. Specific protein product may be detected by Western blot. Preferably, the detection technique will be quantitative or at least semi-quantitative. In other embodiments, the level of collagen breakdown products can be evaluated. For example, the plasma level of MMP-8 protein, functional MMP-8 or collagen breakdown products can be evaluated.

In one embodiment, mRNA is obtained from a sample of cells, and transcripts encoding MMP-8 are detected. To illustrate, an initial crude cell suspension, such as may be obtained from dispersion of a biopsy sample, is sonicated or otherwise treated to disrupt cell membranes so that a crude cell extract is obtained. Known techniques of biochemistry (e.g., preferential precipitation of proteins) can be used for initial purification if desired. The crude cell extract, or a partially purified RNA portion therefrom, is then treated to further separate the RNA. For example, crude cell extract can be layered on top of a 5 ml cushion of 5.7 M CsCl, 10 mM Tris-HCl, pH 7.5, 1 mM EDTA in a 1 in.×3½ in. nitrocellulose tube and centrifuged in an SW27 rotor (Beckman Instruments Corp., Fullerton, Calif.) at 27,000 rpm for 16 hrs at 15° C. After centrifugation, the tube contents are decanted, the tube is drained, and the bottom 0.5 cm containing the clear RNA pellet is cut off with a razor blade. The pellets are transferred to a flask and dissolved in 20 ml 10 mM Tris-HCl, pH 7.5, 1 mm EDTA, 5% sarcosyl and 5% phenol. The solution is then made 0.1 M in NaCl and shaken with 40 ml of a 1:1 phenol:chloroform mixture. RNA is precipitated from the aqueous phase with ethanol in the presence of 0.2 M Na-acetate pH 5.5 and collected by centrifugation. Any other method of isolating RNA from a cellular source may be used instead of this method. Other mRNA isolation protocols, such as the Chomczynski method (described in U.S. Pat. No. 4,843,155), are well known.

The mRNA must be isolated from the source cells under conditions which preclude degradation of the mRNA. The action of RNase enzymes is particularly to be avoided because these enzymes are capable of hydrolytic cleavage of the RNA nucleotide sequence. A suitable method for inhibiting RNase during extraction from cells involves the use of 4 M guanidium thiocyanate and 1 M mercaptoethanol during the cell disruption step. In addition, a low temperature and a pH near 5.0 are helpful in further reducing RNase degradation of the isolated RNA.

In certain embodiments, the next step may be to form DNA complementary to the isolated heterogeneous sequences of mRNA. The enzyme of choice for this reaction is reverse transcriptase, although in principle any enzyme capable of forming a faithful complementary DNA copy of the mRNA template could be used. The cDNA transcripts produced by the reverse transcriptase reaction are somewhat heterogeneous with respect to sequences at the 5′ end and the 3′ end due to variations in the initiation and termination points of individual transcripts, relative to the mRNA template. The variability at the 5′ end is thought to be due to the fact that the oligo-dT primer used to initiate synthesis is capable of binding at a variety of loci along the polyadenylated region of the mRNA. Synthesis of the cDNA transcript begins at an indeterminate point in the poly-A region, and variable length of poly-A region is transcribed depending on the initial binding site of the oligo-dT primer. It is possible to avoid this indeterminacy by the use of a primer containing, in addition to an oligo-dT tract, one or two nucleotides of the RNA sequence itself, thereby producing a primer which will have a preferred and defined binding site for initiating the transcription reaction.

In an exemplary embodiment, there is provided a nucleic acid composition comprising a (purified) oligonucleotide probe including a region of nucleotide sequence which is capable of hybridizing to a sense or antisense sequence of an MMP-8 transcript. The nucleic acid of a cell is rendered accessible for hybridization, the probe is exposed to nucleic acid of the sample, and the hybridization of the probe to the sample nucleic acid is detected. Such techniques can be used to quantitatively determine mRNA transcript levels.

In certain embodiments, detection of the MMP-8 transcripts utilizes a probe/primer in a polymerase chain reaction (PCR) (see, e.g. U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1944) PNAS 91:360-364). In an illustrative embodiment, the method includes the steps of (i) collecting a sample of cells from a patient, (ii) isolating nucleic acid (e.g., mRNA) from the cells of the sample, (iii) contacting the nucleic acid sample (or optionally a cDNA preparation derived therefrom) with one or more primers which specifically hybridize to an MMP-8 transcript under conditions such that hybridization and amplification of at least a portion of the transcript (if present) occurs, and (iv) detecting the presence or absence of an amplification product.

Detection and/or amplification can be carried out with a probe which, for example, hybridizes under stringent conditions to a nucleic acid encoding an MMP-8 transcript. For detection, the probe preferably further comprises a label group attached to the nucleic acid and able to be detected.

In yet another embodiment, the assay detects the presence or absence of the MMP-8 protein in cells of the cell sample, e.g., by determining the level of the MMP-8-inhibitory protein by an immunoassay, gel electrophoresis or the like.

### Polymorphisms

Also within the scope of the invention are (1) sequence variants of the atherosclerosis-associated nucleic acids (e.g., MMP-8 nucleic acids) and (2) methods and tools for detecting such variants. A sequence variant in a nucleic acid can have numerous consequences, e.g., on immune cell physiology. Such genetic alterations can be manifest as 1) a deletion of one or more nucleotides from the nucleic acid; 2) an addition, e.g., insertion, of one or more nucleotides to the nucleic acid; 3) a substitution of one or more nucleotides of the nucleic acid; 4) a chromosomal rearrangement of the nucleic acid; 5) an alteration in the level of a transcript of the nucleic acid (e.g., as a result of a mutation in a transcriptional regulatory region or mRNA stability control region); 6) an aberrant modification of the nucleic acid, such as of the methylation pattern of the genomic DNA; 7) a non-wild type splicing pattern of a messenger RNA transcript of the nucleic acid (e.g., as a result of a mutation in a splicing control region); 8) a non-wild type level of the-protein; 9) an allelic loss of the nucleic acid; and 10) an inappropriate post-translational modification of the protein.

In one aspect, the invention features a method of evaluating a subject, e.g., to identify a predisposition, prescribe a prophylactic, diagnose, or treat the subject. The method includes providing a nucleic acid of the subject; and either a) determining the allelic identity of an atherosclerosis (MMP-8)-associated nucleic acid or b) determining the sequence of at least a nucleotide of the nucleic acid. In a preferred embodiment, the method further includes comparing the allelic identity or sequence to a reference allele or reference sequence of the nucleic acid. The reference allele or reference sequence is associated with an immune disorder or a functional (e.g., normal) immune system. Allelic variants can be detected by a variety of art-known methods. Non-limiting examples include arrays, mismatch cleavage, electrophoretic assays, HPLC assays, and nucleic acid sequencing. The assays can detected nucleotide substitutions, and preferably, also insertions, deletions, translocations, and rearrangements.

Sequence variations in one or more atherosclerosis (MMP-8)-associated nucleic acids can be detected using an array of nucleic acid capture probes, e.g., two-dimensional arrays. Hence, the invention also features an array having a plurality of addresses, each of which is positionally distinguishable from the other. A unique probe is located at each address of the plurality. The array includes at least one address having a probe can be complementary to a region of an atherosclerosis (MMP-8)-associated a nucleic acid, a putative variant (e.g., allelic variant) thereof, or one or more hypothetical variants. In one embodiment, the array includes at least two addresses having a probe for a region of the nucleic acid, one address having a probe substantially complementary to a first allele of the nucleic acid, and one probe substantially complementary to a second allele of the nucleic acid. Optionally, the probe can have one or more mismatches to a region of a nucleic acid, e.g., a destabilizing mismatch at a site other than the query site. Probes with such destabilizing mismatches are considered “substantially complementary” to the target allele, but not to the non-target.

In one embodiment, the array contains multiple probes for the nucleic acid, e.g., four probes for each nucleotide position of the nucleic acid. The array can be used for sequencing by hybridization (U.S. Pat. No. 5,525,464). For example, the array can contain DNA probes synthesized by photolithography (Cronin et al. (1996) Human Mutation 7: 244-255; Kozal et al. (1996) Nature Medicine 2: 753-759).

The array can be designed by first identifying possible mutations in multiple samples, e.g., by sequence by hybridization. Briefly, a first hybridization array of probes can be used to scan through long stretches of DNA in a sample and control to identify base changes between sequences of different samples by making linear arrays of sequential overlapping probes. This step allows the identification of point mutations. This step is followed by a second hybridization array that allows the characterization of specific mutations by using smaller, specialized probe arrays complementary to multiple variants or mutations detected. (see, e.g., Cronin et al., supra) Each mutation is detected with at least a pair of probes, one complementary to the wild-type nucleic acid and the other complementary to the variant nucleic acid. The invention also features oligonucleotides, e.g., nucleotide polymers of 2 to 100 nucleotides in length, which are substantially complementary to an atherosclerosis (MMP-8)-associated a nucleic acid and variants thereof.

In another embodiment, a nucleic acid variant is detected by identifying a mismatched basepair formed by hybridization of a nucleic acid strand of a first allele of the nucleic acid to a nucleic acid strand of a second allele of the nucleic acid. The mismatched basepair can be cleaved, e.g., using one or more proteins that recognize mismatched base pairs in double-stranded DNA (so called “DNA mismatch repair” enzymes). For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662; U.S. Pat. No. 5,459,039).

In still another embodiment, an allelic variant of the nucleic acid is detected as an alteration in electrophoretic mobility. For example, single-strand conformation polymorphism (SSCP) can be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of a query allele and a reference allele of the nucleic acid are denatured and renatured, e.g., together to form a heteroduplex. The secondary structure of single-stranded nucleic acids varies according to sequence; the resulting alteration in electrophoretic mobility enables the detection of even a single base change. The DNA fragments may be labeled or detected with labeled probes. The sensitivity of the assay may be enhanced by using RNA. In a preferred embodiment, variations are detected by electrophoretic separation of double-stranded heteroduplex molecules (Keen et al. (1991) Trends Genet 7:5).

### Nucleic Acid Arrays

Arrays are useful molecular tools for characterizing a sample by multiple criteria. For example, an array having a capture probes for one or more atherosclerosis-associated nucleic acids can be used to diagnose a subject. Arrays can have many addresses, e.g., locatable sites, on a substrate. The featured arrays can be configured in a variety of formats, non-limiting examples of which are described below.

The substrate can be opaque, translucent, or transparent. The addresses can be distributed, on the substrate in one dimension, e.g., a linear array; in two dimensions, e.g., a planar array; or in three dimensions, e.g., a three dimensional array. The solid substrate may be of any convenient shape or form, e.g., square, rectangular, ovoid, or circular. Non-limiting examples of two-dimensional array substrates include glass slides, quartz (e.g., UV-transparent quartz glass), single crystal silicon, wafers (e.g., silica or plastic), mass spectroscopy plates, metal coated substrates (e.g., gold), membranes (e.g., nylon and nitrocellulose), plastics and polymers (e.g., polystyrene, polypropylene, polyvinylidene difluoride, poly-tetrafluoroethylene, polycarbonate, PDMS, nylon, acrylic, and the like). Three-dimensional array substrates include porous matrices, e.g., gels or matrices. Potentially useful porous substrates include: agarose gels, acrylamide gels, sintered glass, dextran, meshed polymers (e.g., macroporous crosslinked dextran, sephacryl, and sepharose), and so forth.

The array can have a density of at least than 10, 50, 100, 200, 500, 1 000, 2 000, 10⁴, 10⁵, 10⁶, 10⁷, 10⁸, or 10⁹ or more addresses per cm² and ranges between. In a preferred embodiment, the plurality of addresses includes at least 10, 100, 500, 1 000, 5 000, 10 000, or 50 000 addresses. In a preferred embodiment, the plurality of addresses includes less than 9, 99, 499, 999, 4 999, 9 999, or 49 999 addresses. Addresses in addition to the address of the plurality can be disposed on the array. The center to center distance can be 5 mm, 1 mm, 100 um, 10 um, 1 um or less. The longest diameter of each address can be 5 mm, 1 mm, 100 um, 10 um, 1 um or less. Each addresses can contain 0 ug, 1 ug, 100 ng, 10 ng, 1 ng, 100 pg, 10 pg, 1 pg, 0.1 pg, or less of a capture agent, i.e. the capture probe. For example, each address can contain 100, 10³, 10⁴, 10⁵, 10⁶, 10⁷, 10⁸, or 10⁹ or more molecules of the nucleic acid.

Arrays can be fabricated by a variety of methods, e.g., photolithographic methods (see, e.g., U.S. Pat. Nos. 5,143,854; 5,510,270; and 5,527,681), mechanical methods (e.g., directed-flow methods as described in U.S. Pat. No. 5,384,261), pin based methods (e.g., as described in U.S. Pat. No. 5,288,514), and bead based techniques (e.g., as described in PCT US/93/04145). The capture probe can be a single-stranded nucleic acid, a double-stranded nucleic acid (e.g., which is denatured prior to or during hybridization), or a nucleic acid having a single-stranded region and a double-stranded region. Preferably, the capture probe is single-stranded. The capture probe can be selected by a variety of criteria, and preferably is designed by a computer program with optimization parameters. The capture probe can be selected to hybridize to a sequence rich (e.g., non-homopolymeric) region of the nucleic acid. The Tₘ of the capture probe can be optimized by prudent selection of the complementarity region and length. Ideally, the Tₘ of all capture probes on the array is similar, e.g., within 20, 10, 5, 3, or 2° C. of one another. A database scan of available sequence information for a species can be used to determine potential cross-hybridization and specificity problems.

The isolated nucleic acid is preferably mRNA that can be isolated by routine methods, e.g., including DNase treatment to remove genomic DNA and hybridization to an oligo-dT coupled solid substrate (e.g., as described in Current Protocols in Molecular Biology, John Wiley & Sons, N.Y). The substrate is washed, and the mRNA is eluted.

The isolated mRNA can be reversed transcribed and optionally amplified, e.g., by rtPCR, e.g., as described in (U.S. Pat. No. 4,683,202). The nucleic acid can be an amplification product, e.g., from PCR (U.S. Pat. No. 4,683,196 and 4,683,202); rolling circle amplification (“RCA,” U.S. Pat. No. 5,714,320), isothermal RNA amplification or NASBA (U.S. Pat. Nos. 5,130,238; 5,409,818; and 5,554,517), and strand displacement amplification (U.S. Pat. No. 5,455,166). The nucleic acid can be labeled during amplification, e.g., by the incorporation of a labeled nucleotide. Examples of preferred labels include fluorescent labels, e.g., red-fluorescent dye Cy5 (Amersham) or green-fluorescent dye Cy3 (Amersham), and chemiluminescent labels, e.g., as described in U.S. Pat. No. 4,277,437. Alternatively, the nucleic acid can be labeled with biotin, and detected after hybridization with labeled streptavidin, e.g., streptavidin-phycoerythrin (Molecular Probes).

The labeled nucleic acid can be contacted to the array. In addition, a control nucleic acid or a reference nucleic acid can be contacted to the same array. The control nucleic acid or reference nucleic acid can be labeled with a label other than the sample nucleic acid, e.g., one with a different emission maximum. Labeled nucleic acids can be contacted to an array under hybridization conditions. The array can be washed, and then imaged to detect fluorescence at each address of the array.

The expression data can be stored in a database, e.g., a relational database such as a SQL database (e.g., Oracle or Sybase database environments). The database can have multiple tables. For example, raw expression data can be stored in one table, wherein each column corresponds to a nucleic acid being assayed, e.g., an address or an array, and each row corresponds to a sample. A separate table can store identifiers and sample information, e.g., the batch number of the array used, date, and other quality control information.

Expression profiles obtained from nucleic acid expression analysis on an array can be used to compare samples and/or cells in a variety of states as described in Golub et al. ((1999) Science 286:531). In one embodiment, multiple expression profiles from different conditions and including replicates or like samples from similar conditions are compared to identify nucleic acids whose expression level is predictive of the sample and/or condition. Each candidate nucleic acid can be given a weighted “voting” factor dependent on the degree of correlation of the nucleic acid's expression and the sample identity. A correlation can be measured using a Euclidean distance or the Pearson correlation coefficient.

The similarity of a sample expression profile to a predictor expression profile (e.g., a reference expression profile that has associated weighting factors for each nucleic acid) can then be determined, e.g., by comparing the log of the expression level of the sample to the log of the predictor or reference expression value and adjusting the comparison by the weighting factor for all nucleic acids of predictive value in the profile.

Nucleic acids of all categories can be used to characterize a sample. In a preferred embodiment, the magnitude of change is determined and used for more sophisticated classification, e.g., with quantitative boundaries. As described above, such characterization is best determined using quantitative metrics and algorithms.

### Polypeptide Arrays

The expression level of a polypeptide encoded by an atherosclerosis-associated nucleic acid can be determined using an antibody specific for the polypeptide (e.g., using a Western blot or an ELISA assay). Moreover, the expression levels of multiple polypeptides encoded by these nucleic acids can be rapidly determined in parallel using a polypeptide array having antibody capture probes for each of the polypeptides. Antibodies specific for a polypeptide can be generated by a method described herein (see “Antibodies”).

A low-density (96 well format) protein array has been developed in which proteins are spotted onto a nitrocellulose membrane Ge, H. (2000) Nucleic Acids Res. 28, e3, I-VII). A high-density protein array (100,000 samples within 222×222 mm) used for antibody screening was formed by spotting proteins onto polyvinylidene difluoride (PVDF) (Lueking et al. (1999) Anal. Biochem. 270, 103-111). Polypeptides can be printed on a flat glass plate that contained wells formed by an enclosing hydrophobic Teflon mask (Mendoza, et al. (1999). Biotechniques 27, 778-788.). Also, polypeptide can be covalently linked to chemically derivatized flat glass slides in a high-density array (1600 spots per square centimeter) (MacBeath, G., and Schreiber, S. L. (2000) Science 289, 1760-1763). De Wildt et al., describe a high-density array of 18,342 bacterial clones, each expressing a different single-chain antibody, in order to screening antibody-antigen interactions (De Wildt et al. (2000). Nature Biotech. 18, 989-994). These art-known methods and other can be used to generate an array of antibodies for detecting the abundance of polypeptides in a sample. The sample can be labeled, e.g., biotinylated, for subsequent detection with streptavidin coupled to a fluorescent label. The array can then be scanned to measure binding at each address.

The nucleic acid and polypeptide arrays of the invention can be used in wide variety of applications. For example, the arrays can be used to analyze a patient sample. The sample is compared to data obtained previously, e.g., known clinical specimens or other patient samples. Further, the arrays can be used to characterize a cell culture sample, e.g., to determine a cellular state after varying a parameter, e.g., exposing the cell culture to an antigen, a transgene, or a test compound.

### Methods for Evaluating a Sample

In another aspect, the invention features a method of evaluating a sample, which includes the following steps. A physician obtains a sample (i.e., “patient sample”), e.g., a blood sample, from the patient. The patient sample can be delivered to a diagnostics department which can collate information about the patient, the patient sample, and results of the evaluation. A courier service can deliver the sample to a diagnostic service. Location of the sample is monitored by a courier computer system, and can be tracked by accessing the courier computer system, e.g., using a web page across the Internet. At the diagnostic service, the sample is processed to produce a sample expression profile. For example, nucleic acid is extracted from the sample, optionally amplified, and contacted to a nucleic acid microarray. Binding of the nucleic acid to the microarray is quantitated by a detector that streams data to the array diagnostic server. The array diagnostic server processes the microarray data, e.g., to correct for background, sample loading, and microarray quality. It can also compare the raw or processed data to a reference expression profile, e.g., to produce a difference profile. The raw profiles, processed profiles and/or difference profiles are stored in a database server. A network server manages the results and information flow. In one embodiment, the network server encrypts and compresses the results for electronic delivery to the healthcare provider's internal network. The results can be sent across a computer network, e.g., the Internet, or a proprietary connection. For data security, the diagnostic systems and the healthcare provider systems can be located behind firewalls. In another embodiment, an indication that the results are available can also be sent to the healthcare provider and/or the patient, for example, by to an email client. The healthcare provider, e.g., the physician, can access the results, e.g., using the secure HTTP protocol (e.g., with secure sockets layer (SSL) encryption). The results can be provided by the network server as a web page (e.g., in HTML, XML, and the like) for viewing on the physician's browser.

Further communication between the physician and the diagnostic service can result in additional tests, e.g., a second expression profile can be obtained for the sample, e.g., using the same or a different microarray.

### Transgenic Animals

The invention provides non-human transgenic animals. Such animals are useful for studying the function and/or activity of a MMP-8 protein and for identifying and/or evaluating modulators of MMP-8 activity. As used herein, a “transgenic animal” is a nonhuman animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which preferably is integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous MMP-8 gene has been altered by, e.g., by homologous recombination between the endogenous gene and an exogenous DNA molecule introduced into a cell of the animal, e.g., an embryonic cell of the animal, prior to development of the animal.

Intronic sequences and polyadenylation signals can also be included in the transgene to increase the efficiency of expression of the transgene. A tissue-specific regulatory sequence(s) can be operably linked to a transgene of the invention to direct expression of a MMP-8 protein to particular cells. A transgenic founder animal can be identified based upon the presence of a MMP-8 transgene in its genome and/or expression of MMP-8 mRNA in tissues or cells of the animals. A transgenic founder animal can then be used to breed additional animals carrying the transgene. Moreover, transgenic animals carrying a transgene encoding a MMP-8 protein can further be bred to other transgenic animals carrying other transgenes.

MMP-8 proteins or polypeptides can be expressed in transgenic animals or plants, e.g., a nucleic acid encoding the protein or polypeptide can be introduced into the genome of an animal. In preferred embodiments the nucleic acid is placed under the control of a tissue specific promoter, e.g., a milk or egg specific promoter, and recovered from the milk or eggs produced by the animal. Suitable animals are mice, pigs, cows, goats, and sheep.

The invention also includes a population of cells from a transgenic animal.

The invention now being generally described, it will be more readily understood by reference to the following examples which are included merely for purposes of illustration of certain aspects and embodiments of the present invention, and are not intended to limit the invention. All publications cited herein are incorporated in their entirety by reference.

### EXAMPLES

The experimental procedures described herein are used in Examples 1-3.

### Materials

Human recombinant IL-1β and TNFα were obtained from Endogen (Cambridge, Mass.), Escherichia coli endotoxin (LPS) from Sigma (St. Louis, Mo.), recombinant MMP-8 from Chemicon (Tenecula, Calif.), and recombinant CD40L from Leinco Technologies (St. Louis, Mo.). Mouse monoclonal and rabbit polyclonal antibodies against human MMP-8 were obtained from Calbiochem (La Jolla, Calif.) and Chemicon, respectively. Control mAb and rabbit Ig employed in immunohistochemistry were obtained from PharMingen (La Jolla, Calif.).

### Cell Isolation and Culture

Human vascular endothelial cells (EC) and smooth muscle cells (SMC) were isolated from saphenous veins by collagenase treatment (1 mg/ml; Worthington Biochemicals, Freehold, N.J.) and explant outgrowth, respectively, and cultured as described in (Sukhova G K et al. Circulation 1999; 99: 2503-9; Schonbeck U et al. Circ Res. 1997; 8 1: 448-54; Schonbeck U et al. J Exp Med. 1999; 189: 843-53). Both cell types were cultured before (24 h) and during the experiment in media lacking FBS, as described in (Schonbeck U et al. J Exp Med. 1999; 189: 843-53). EC in M 99 supplemented with 0.1% human serum albumin; SMC in IT (Insulin/Transferrin) medium. Culture media and FBS contained less than 40 pg endotoxin/ml as determined by the chromogenic Limulus amoebocyte assay (QLC-1000; BioWhittaker).

Mononuclear phagocytes were isolated from freshly prepared human peripheral blood mononuclear cells (PBMC) by density gradient centrifugation, employing Lymphocyte Separation Medium (Organon-Teknika, Durham, N.C.), and subsequent adherence to plastic culture flasks. Mononuclear phagocytes were used directly (monocytes) for the experiments or cultured for 1, 3, or 11 days (macrophages, MØ) in RPM1 1640 containing 2% human serum (Sigma). Before (24 h) and during stimulation, the cells were cultured in RPM1 1640 lacking serum. The purity of monocytes/macrophages was ≧92%, as determined by FACS analysis (anti-human CD68 mAb FITC, PharMingen).

### RNA Isolation

Total RNA was isolated from monocyte-derived macrophages employing RNazol (Tel-Test; Friendswood, Tex.). Employing Superscript Reverse Transcriptase (GibcoBRL), total RNA (10 μg) was reverse transcribed to obtain the oligo-dT30 primed, [α³³P]dCTP-labelled first-strand cDNA probe. Hybridization experiments were performed following standard techniques. Quadruplicate filters per probe were pre-hybridized (65° C., 1 h) in 10% formamide-Church Buffer containing Salmon sperm DNA (10 mg/ml) and subsequently hybridized (18 h) with the respective probe. Filters were washed twice (65° C., 15 min) with 2×SSC/1% SDS and 0.1×SSC/O. 5% SDS, respectively, rinsed in 2×SSC at room temperature, and baked (2 h, 85° C.). Finally, dried filters were exposed (3-5 days) on phospho-imaging plates (Fuji-Film), and median±SD of quadruplicate filters were calculated. Treatment with CD40L was normalized to the respective time point of untreated control.

### Western Blot Analysis

Tissue extracts (50 μg total protein/lane) obtained from frozen nonatherosclerotic arteries or atheromatous carotid plaques, as well as cell culture lysates (20 μg total protein/lane) and supernatants (50 μl) were separated by standard SDS-PAGE under reducing conditions and applied to Western blot analysis as described previously. Immunoreactive proteins were visualized using the Western blot chemiluminescence system (NEN™, Boston, Mass.).

### In situ Hybridization

In situ hybridization was performed accordingly to the instructions of the manufacturer (Biogenex, San Ramos, Calif.). Frozen tissue sections were fixed in cold acetone, air-dried and incubated (10 min, 65° C.; subsequently 2 h, 37° C.) with a mixture of FITC-labeled MMP-8

(5′-TCGACAGTCTCCGACTCCATCTTTCTCGAT-3′;

5′-CGGAACGACAGAGGGTTGATACGAAAGTCC-3′;

5′-TTGTATGAAGAAACATTTACTGGTTAA GAC3′;

5′-TCTTGATCTAAAACCAATCTTCATTCCTAA-3′) or random (control) oligomers in hybridization-buffer (30% formamide, 0.6 M NaCl₂, 10% dextran sulfate, 50 mM Tris (pH 7.5), 0.1% Sodium-pyro-phosphate, 0.2% Ficoll, 5 mM EDTA). Finally, slides were washed 3 times and stained with alkaline phosphatase-conjugated rabbit Fab′ anti-FITC (30 min) and NBT/BCIP chromogen solution (1 h).

### Immunohistochemistry

Serial cryostat sections (5 μm) of surgical specimens of human carotid atheroma and aorta were cut, air dried onto microscope slides, fixed in acetone (−20° C., 5 min), and preincubated with PBS containing 0.3% hydrogen peroxide. Subsequently, sections were incubated (30 min) with primary or control (mouse myeloma protein MOPC-21, Sigma) antibody, diluted in PBS supplemented with 5% appropriate serum, and processed according to the recommendations provided by the supplier (Universal Dako LSAB Kit, Dako Co.).

For colocalization of MMP-8 with the respective cell type, anti-human MMP-8 antibody (1:400) was applied (90 min), followed by biotinylated secondary antibody (45 min) and Texas red-conjugated streptavidin (Amersham, Arlington Heights, IL) (20 min). Subsequent to application of the avidin/biotin blocking kit (Vector), anti-muscle actin mAb for SMC (1:200; Enzo Diagnostics, New York, N.Y.), anti-CD3 1 mAb for EC (1: 35, Dako), or anti-CD68 mAb for macrophages (1: 500, Dako) were added and sections incubated overnight (4° C.). Subsequently, biotinylated horse-anti-mouse secondary antibodies were applied (45 min), followed by Streptavidin-FITC (Amersham) (20 min). Staining of collagen type I and III employed Picrosirius red, as described previously. Cleaved interstitial type I collagen was detected by staining with a polyclonal rabbit antibody reactive with the COL3/4Cₛₕₒᵣₜ, neoepitope (Sukhova G K et al. Circulation 1999 99: 2503-9).

For double immunofluorescence labeling for MMP-8 with cleaved or intact type I collagen, frozen sections were incubated 90 min with rabbit-anti-human COL3/4Cₛₕₒᵣₜ or mouse-anti-human type I collagen antibody, followed by biotinylated secondary antibody (45 min, Vector Laboratories) and FITC-conjugated streptavidin (30 min; Amersham Corp.). Subsequently, specimens were treated with an avidin/biotin blocking kit (Vector Laboratories), washed, and stained with mouse-anti-human MMP-8 antibody (overnight, 4° C.), biotinylated secondary horse-anti-mouse antibody, and streptavidin conjugated with Texas red (Amersham Corp.). Nuclei were stained with bisbenzimide (Calbiochem).

### Example 1

### Expression of MMP-8 in Human Atheroma-associated Cells in vitro

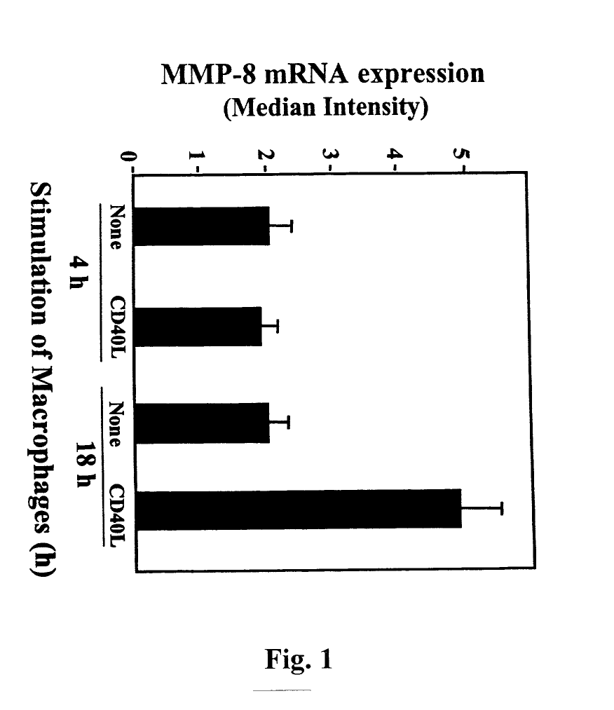

Transcriptional profiling analysis demonstrated that stimulation of monocyte-derived macrophages with CD40 ligand (CD40L) enhanced the expression of MMP-8 transcripts (FIG. 1). The experimental conditions are briefly set forth as follows. Total RNA preparations were obtained from unstimulated or CD40 ligand-(10 μg/ml) stimulated (4 and 18 hrs, respectively) macrophages, derived by culture for ten days of mononuclear phagocytes, and were applied to transcriptional profiling analysis. The median values of quadruplicate filters per probe hybridization are given. Error bars represent standard deviation. Intensity values of cDNA spots obtained in cultures treated with CD40L were normalized to the respective time point of untreated control. Comparable data were obtained with mononuclear phagocytes from three different donors.

In accordance with the data obtained for the transcriptional regulation, unstimulated cultures of EC, SMC, and mononuclear phagocytes did not express MMP-8 protein constitutively. However, stimulation with proinflammatory cytokines, e.g., IL-1β or CD40L, as well as TNFα or LPS, induced expression and release of immunoreactive MMP-8 in all three cell types. Atheroma-associated cells released two major MMP-8 bands migrating at approximately 75 kDa and 45 kDa, respectively, the expected molecular weights reported for this enzyme's latent and active form. EC culture supernatants expressed only a single band at approximately 75 kDa. In contrast to polymorphonuclear neutrophils, resting EC, SMC, or macrophages did not contain cell-associated MMP-8. Accumulation of the enzyme within cell lysates required stimulation with pro-inflammatory cytokines, e.g., IL-β or CD40L, suggesting that activation triggered de novo synthesis and release of MMP-8 in these cell types. The following experimental conditions were used. Culture supernatants or lysates (50 ug) were obtained from unstimulated, IL-1β (10 ng/ml), or CD40L (10 μg/ml) stimulated (24 h) EC, SMC, monocyte-derived macrophages), or polymorphonuclear granulocytes (PMN), and were analyzed by Western blotting for expression of MMP-8 immunoreactive proteins. Comparable data were obtained employing cells from at least three different donors.

Since macrophages constitute a major source of matrix-degrading proteinases, particularly interstitial collagenases (Sukhova G K et al. Circulation 1999 99: 2503-9) within human atheroma, we further analyzed whether differentiation of freshly isolated peripheral blood mononuclear phagocytes into monocyte-derived macrophages affected the expression of MMP-8. Briefly, culture supernatants of unstimulated, IL-1β-(10 ng/ml), or CD40 ligand-(10 μg/ml) stimulated (24 h) mononuclear phagocytes cultured for 0, 1, 3, 11 days, were analyzed by Western blotting for expression of MMP-8 immunoreactive proteins. Comparable data were obtained with cells from three different donors. Interestingly, culture supernatants of either unstimulated, IL-1β-, or CD40L-stimulated freshly isolated mononuclear phagocytes did not contain immunoreactive MMP-8. However, prolonged culture yielded low basal expression of MMP-8, which increased substantially upon stimulation with either IL-1β or CD40L.

### Example 2

### Expression of MMP-8 in Human Atheroma-associated Cells in situ

Given the inducibility of MMP-8 expression in atheroma-associated cells in vitro, MMP-8 transcript and protein expression was determined in EC, SMC, and macrophages within human atherosclerotic lesions in situ. In contrast to non-diseased arterial tissue, human atherosclerotic lesions expressed MMP-8 mRNA abundantly, particularly in macrophages within the shoulder region, the prototypical site of plaque rupture. Staining for MMP-8 further colocalized with the endothelium and the SMC-enriched fibrous cap. Briefly, serial cryostat sections from atherosclerotic carotid atheroma and nonatherosclerotic aortae were analyzed for MMP-8 transcript expression by in situ hybridization. Higher magnifications demonstrated localization of MMP-8 transcripts within the luminal endothelium, the SMC-enriched fibrous cap, and the macrophage-enriched shoulder region. Scrambled oligomers of identical size were employed as negative control. Analysis of non-diseased arteries and surgical specimens of atheroma from three different donors showed similar results.

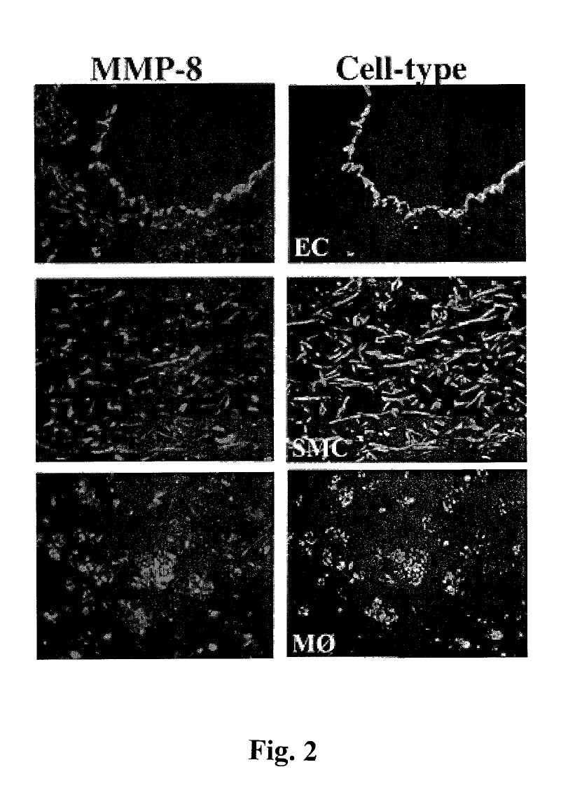

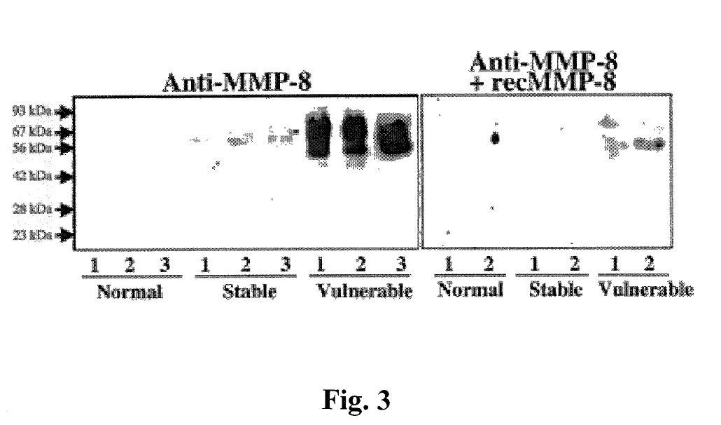

In accordance with the expression of MMP-8 transcript, immunhistochemical analysis demonstrated expression of the MMP-8 protein in atherosclerotic, but not non-diseased arterial tissue. As in the in situ hybridization studies, MMP-8 protein accumulated predominantly within the shoulder region of the atherosclerotic plaque. Colocalization of the enzyme with all three atheroma-associated cell types, EC, SMC, and macrophages, was formally demonstrated by immunofluorescence double labeling (see FIG. 2). Interestingly, atherosclerotic lesions characterized by features associated with rupture-prone plaques, e.g., large lipid core and thin fibrous cap, expressed more immunoreactive MMP-8, compared to less vulnerable appearing (‘stable’) lesions (see FIG. 3). Extracts of advanced atherosclerotic lesions contained significantly more immunoreactive MMP-8 than did plaques with more stable morphology or non-atherosclerotic tissue.

The following experimental conditions were used. To visualize expression of MMP-8 protein in human atherosclerotic lesions, serial cryostat sections from non-atherosclerotic aortae and atherosclerotic carotid atheroma, dichotomized by features associated with either ‘stable’ or ‘vulnerable’ lesions, were analyzed for the expression of MMP-8, as well as smooth muscle α-actin or CD68 (macrophages). Analysis of non-diseased arteries, ‘stable’, and vulnerable surgical specimen of atheroma from three different donors showed similar results. To detect expression of MMP-8 protein in human atherosclerotic lesions, protein extracts (50 μg) obtained from frozen tissue of three different donors of non-atherosclerotic carotid arteries, as well as carotid plaques, dichotomized into lesions characterized by features associated with ‘stable’ or ‘vulnerable’ plaques, were analyzed by Western blotting employing anti-MMP-8 antibody. For colocalization studies with human vascular EC, SMC, as well as macrophages in human atherosclerotic lesions, double-immunofluorescence staining was utilized. MMP-8 colocalized with EC (anti-CD31), SMC (anti-α-actin) or macrophages (anti-CD68) within the shoulder region of atherosclerotic plaques. Analysis of surgical specimens of atheroma from three different donors showed similar results.

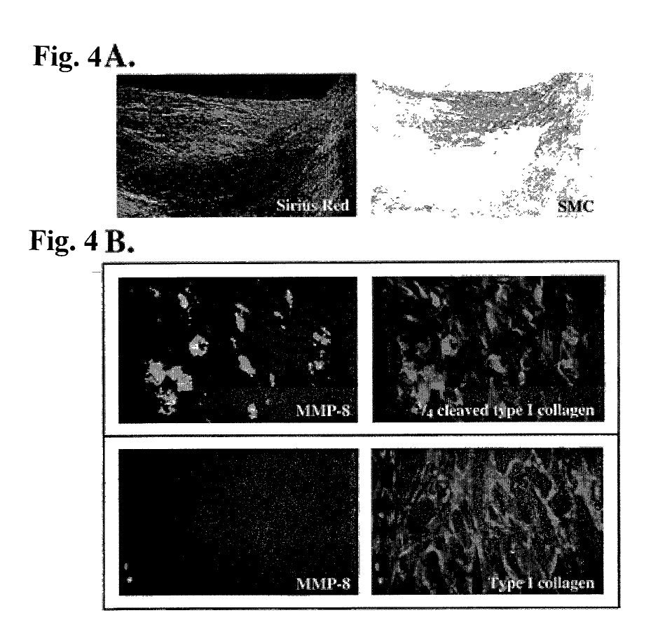

Previous studies have provided direct evidence for collagenolysis within the shoulder region of ‘vulnerable’ atherosclerotic plaques. As shown above, this is the site of prominent MMP-8 expression (Sukhova G K et al. Circulation 1999 99: 2503-9). Therefore, colocalization of MMP-8 with its preferred substrate, type I collagen, as well as the initial three-quarter-length breakdown product was analyzed (see FIG. 4). Indeed, immunofluorescence double-labeling colocalized the enzyme with degraded type I collagen and showed an inverse correlation with staining for intact type I collagen. The following experimental conditions were used: Picrosirius red staining identified collagen expression within human atherosclerotic lesions and immunofluorescence double labeling identified cleaved, three quarter length fragments and intact type I collagen. Double-immunofluorescence staining was utilized to colocalize MMP-8 with either intact type I collagen or cleaved, three-quarter-length fragments of type I collagen (within the shoulder region of atherosclerotic plaques. Analysis of surgical specimens of atheroma from two different donors showed similar results.

### Example 3

### A Novel Pathological Role for MMP-8

Degradation of extracellular matrix macromolecules by matrix degrading enzymes, such as MMP, influences the evolution of an atherosclerotic lesion towards vulnerable, rupture-prone plaques. Since interstitial collagen, i.e. type I collagen, comprises the major load-bearing molecule within the plaques fibrous cap overlying the pro-coagulant lipid core, collagenolysis in advanced atherosclerotic lesions probably promotes the evolution of rupture-prone lesions (Morton L F et al. Atherosclerosis 1982; 42: 41-51; Rekhter M et al. Am. J. Pathol. 1993; 143: 1634-1648; Stary H C, Eur Heart J. 1990; 11 Suppl E: 3-19). Recently, direct evidence showed MMP-mediated collagenolysis of type I collagen within human atheroma at the prominent site of MØ accumulation, as well as of MMP-1 and MMP-13 expression (Sukhova G K et al. Circulation 1999 99: 2503-9; Nikkari S T et al. Circulation. 1995 92: 1393-9). However, each of the known interstitial collagenases has distinct preferences for different types of fibrillar collagen, with MMP-1 preferably processing type III collagen and MMP-13 preferably processing type III collagen. Interestingly, MMP-8 preferentially processes type I collagen (Horwitz A L et al. Proc Natl Acad Sci U S A. 1977; 74: 897-901; Hasty K A et al. J Biol Chem. 1987; 262: 10048-52; Welgus H G, et al. J Biol Chem. 1981; 256: 951 1-5). Due to the traditional designation of MMP-8 as a neutrophil enzyme, the role of MMP-8 in atherogenesis has been neglected. The unbiased survey afforded by transcriptional profiling shown above pointed to a role of this enzyme in atherogenesis, despite its name. Curiously, recent reports have suggested expression of MMP-8 by cells other than neutrophils, including rheumatoid synovial fibroblasts and EC and articular chondrocytes (Hanemaaijer R et al. J Biol Chem. 1997; 272: 31504-9; Cole A A et al. J. Biol Chem. 1996 27 1: 11023-6). The surprising finding that EC, SMC, and macrophages within human atherosclerotic lesions express MMP-8 affirms that the expression of this interstitial collagenase extends beyond a single cell type. The cytokine-induced expression of MMP-8 in EC, SMC, and macrophages, differs from the situation in the traditional source, the neutrophil, which contains preformed MMP-8 (Weiss S J, et al. Science. 1985 227: 747-9; Hasty K A et al. J Biol Chem. 1986 261: 5645-50; Mookhtiar K A et al. Biochemistry. 1990 29: 10620-7). Thus, in chronic inflammation, cells such as EC, SMC, and MØ release MMP-8. In acute inflammation associated with PMN infiltration, MMP release can be immediate. In view of the role of hypochlorous acid in MMP-8 activation (Weiss S J, et al. Science. 1985 227: 747-9), it is noteworthy that macrophages in advanced atherosclerotic lesions contain myeloperoxidase (Daugherty A et al. J Clin Invest. 1994 94: 437-44), the enzyme responsible for hypochlorous acid production.

Colocalization with cleaved, but not intact, type I collagen indicates a prominent role for MMP-8 in the loss of this major load-bearing molecule in human atheroma. The degradation of type I collagen might prove critical in the advanced rather than early atherosclerotic lesion, since loss of extracellular matrix characterizes lesion progression towards vulnerable, rupture-prone plaques. Our current finding of enhanced MMP-8 expression in lesions of “unstable” morphology agrees with this model.

The surprising finding that human vascular EC, SMC, and macrophages express and release mature interstitial collagenase MMP-8 upon stimulation in vitro and in situ not only broadens knowledge of the expression pattern of this ‘neutrophil collagenase’, but further suggests a novel pathological role of MMP-8. Designing inhibitors of MMPs of restricted specificity may obviate some of the toxicity encountered in clinical trials of broad spectrum agents. The present identification of a likely role for MMP-8 in atherogenesis thus has practical therapeutic as well as theoretic implications.

High expression of MMP-8 has also been observed in tissue samples obtained from patients suffering from chronic obstructive pulmonary disease and inflammatory bowel disease. The role of MMP-8, and the associated degradation of type I collagen, may play an important role in many non-neutrophil mediated inflammatory disorders.

### Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A method of inhibiting the activity, processing, translation, or expression of matrix metalloprotease-8 (MMP-8), comprising contacting MMP-8, or an MMP-8-expressing cell, with an agent, in an amount sufficient to inhibit the activity, expression or processing of MMP-8.

2. A method of treating or preventing, in a subject, a disorder characterized by aberrant expression, activity or processing of MMP-8 in a macrophage, endothelial cell, or smooth muscle cell, said method comprises administering to the subject an agent that inhibits the activity, processing, translation, or expression of MMP-8 in an amount effective to treat or prevent the disorder.

3. A method of treating or preventing a cardiovascular disorder in a subject, comprising administering to the subject an agent that inhibits the activity, processing, translation, or expression of MMP-8 in an amount effective to treat or prevent the cardiovascular disorder.

4. A method of treating or preventing an endothelial cell disorder in a subject, comprising administering to the subject an agent that inhibits the activity, processing, translation, or expression of MMP-8 in an amount effective to treat or prevent the disorder.

5. The method of claim 1, wherein the agent is an MMP-8 specific inhibitor.

6. The method of any of claims 2, 3, or 4, wherein the agent is an MMP-8 specific inhibitor.

7. The method of claim 5, wherein the MMP-8-specific inhibitor is selected from the group consisting of an anti-MMP-8 antibody, a small molecule inhibitor, a peptide, and a collagen I fragment.

8. The method of claim 1, wherein the MMP-8-expressing cell is an atheroma-associated cell selected from the group consisting of an endothelial cell, a smooth muscle cell and a macrophage.

9. The method of claim 2, wherein the disorder is selected from the group consisting of atherosclerosis, myocardial infarction, aneurism, and stroke.

10. The method of claim 2, wherein the subject is a human suffering from, or at risk for, atherosclerosis.

11. The method of claim 10, wherein the subject is a human suffering from, or at risk for, the rupture of an atherosclerostic plaque.

12. The method of claim 2, wherein the agent is administered in combination with a non-specific matrix metalloprotease inhibitor.

13. The method of claims 3, wherein the agent is administered in combination with a cholesterol-lowering agent.

14. The method of claim 3, wherein the agent is administered in combination with an interventional procedure.

15. The method of claim 14, wherein the interventional procedure is selected from the group consisting of angioplasty, placement of a shunt, stent, synthetic or natural excision grafts, indwelling catheter, valve and other implantable devices.

16. The method of claim 2, wherein the agent, alone or in combination with the second agent or procedure, inhibits one or more of: atherosclerotic lesion formation; development or rupture; lipid accumulation; degradation of type I, II, or III collagen; or rupture of atherosclerotic plaques.

17. A method for evaluating the efficacy of a treatment of a cardiovascular disorder, or an endothelial cell disorder, in a subject, comprising:
evaluating the expression of MMP-8 nucleic acids or polypeptides, wherein a decrease in the level of MMP-8 nucleic acids or polypeptides in a sample obtained after treatment, relative to the level of expression in a similar sample before treatment, is indicative of the efficacy of the treatment of said disorder.

18. A method of evaluating, or identifying, an agent for the ability to inhibit the activity, processing, translation, or expression of an MMP-8 nucleic acid or protein, comprising:
providing a test agent, an MMP-8 protein or a cell expressing MMP-8; and an MMP-8 substrate;
contacting said test agent, said MMP-8 protein or cell expressing MMP-8, and said MMP-8 substrate, under conditions that allow an interaction between said MMP-8 protein and said MMP-8 substrate to occur; and
determining whether said test agent inhibits the interaction between said MMP-8 protein and said MMP-8 substrate,
wherein a decrease in the amount of interaction between said MMP-8 protein and said MMP-8 substrate in the presence of the test agent, relative to the interaction in the absence of the test agent, is indicative of inhibition of the activity, processing, translation, or expression of an MMP-8 nucleic acid or protein.

19. The method of claim 18, which further comprises the step of evaluating the test agent in an atheroma-associated cell, in a subject, to thereby determine the effect of the test agent on the activity, processing, translation, or expression of the MMP-8 nucleic acid or protein.

20. The method of claim 18, wherein the test agent is an MMP-8-specific inhibitor.

21. The method of claim 20, wherein the test agent is a peptide, a small molecule, a member of a combinatorial library, or an antibody.

22. The method of claim 20, wherein the test agent is a dsRNA molecule, an antisense RNA molecule, a ribozyme, or a triple helix molecule.
